SEQUENCE LISTING (14 sequences)

SEQ ID NO: 1 - protein, 575 aa, unidentified [sequence omitted]

SEQ ID NO: 2 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKRKIHTVIYDSLKKLPFPVKKIAKDFKLTVLKGDIDYHKERPVGYKITPEEYAYIKNDIQIIAEALKLMFDQGLDRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 3 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKHKIHTVIYDSLKKLPFPVKKIAKDFKLTVLKGDIDYHKERPVGYKITPEEYAYIKNDIQIIAEALKLMFDQGLDRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 4 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKRKIHTLIYDSLKKLPFPVKKIAKDFKLTVLKGDIDYNKERPVGYKITPEEYAYIKNDIQIIAEALKLMFDQGLDRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 5 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKRKIHTVIYDSLKKLPFPVKKIAKDFKLTVLKGNIDYLKERPVGYKITPEEYAYIKNDIQIIAEALKLMFDQGLDRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 6 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKHKIHTSIYDSLKKLPFPVKKIAKDFKLTVLKGNIDYHKERPVGYKITPEEYAYIKNDIQIVAEALKLMFDQGLDRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 7 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKRKIHTVIYDSLKKLPFPVKKIAKDFKLTVLKGDIDYHKERPVGYKITPEEYAYIKNDILIIAEALKLMFDQGLDRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 8 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKRKIHTSIYDSLKKLPFPVKKIAKDFKLTVLKGDIDYHKERPVGYKITPEEYAYIKNDILIIAEALKLMFDQGLTRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 9 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKRKIHTVIYDSLKKLPFPVKKIAKDFKLTVLKGDIDYHKERPVGYKITPEEYAYIKNDIQIVAEALKLMFDQGLDRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 10 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKRKIHTVIYDSLKKLPFPVKKIAKDFKLTVLKGDIDYLKERPVGYKITPEEYAYIKNDIQIIAEALKLMFDQGLTRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 11 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKRKIHTVIYDSLKKLPFPVKKIAKDFKLTVLKGVIDYHKERPVGYKITPEEYAYIKNDIQIIAEALKLMFDQGLTRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 12 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKHKIHTVIYDSLKKLPFPVKKIAKDFKLTVLKGDIDYLKERPVGYKITPEEYAYIKNDILIVAEALKLMFDQGLDRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 13 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKRKIHTSIYDSLKKLPFPVKKIAKDFKLTVLKGNIDYHKERPVGYKITPEEYAYIKNDIQIIAEALKLMLDQGLDRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 14 - protein, 575 aa, unidentified
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICIGYKGKRKIHTSIYDSLKKLPFPVKKIAKDFKLTVLKGDIDYHKERPVGYKITPEEYAYIKNDIQIIAEALKLMLDQGLDRMTAGSDSLKGFKDIITTKKFKKVFPTLSLGLDKEVRKAYRGGFTWLNDKFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELLKEHYKLYNVEYVRGLKFKATTGLFKDFIDKWYYIKTHSEGAKKQLAKLMLNSLYGKFGSNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

(19) United States
(12) Patent Application Publication
(10) Pub. No.: US 2026/0049293 A1
(43) Pub. Date: Feb. 19, 2026

(54) DNA POLYMERASE AND METHODS OF USE THEREOF

(71) Applicant: Pando Bioscience, Inc., Watertown, MA (US)

(72) Inventors: Yangxiaolu CAO, Watertown, MA (US); Lucas CAREY, Watertown, MA (US); Natali OZBER, Watertown, MA (US); Manjodh SINGH, Watertown, MA (US); Yang WANG, Watertown, MA (US)

(73) Assignee: Pando Bioscience, Inc., Watertown, MA (US)

(21) Appl. No.: 19/297,824

(22) Filed: Aug. 12, 2025

Related U.S. Application Data
(60) Provisional application No. 63/682,576, filed on Aug. 13, 2024.

(51) Int. Cl. C12N 9/12; C12Q 1/6844

(52) U.S. Cl. CPC C12N 9/1252; C12Q 1/6844; C12Y 207/07007

(57) ABSTRACT

In an aspect, provided is a DNA polymerase with an amino acid sequence of at least 80% sequence identity with SEQ ID NO: 1 and having one or more amino acid substitution, wherein the one or more amino acid substitution is selected from the group consisting of A83S, N91I, R96H, R96C, M97V, G108D, G111V, R113H, V118S, S122N, L126I, P127L, A134T, D145V, D145N, D147V, H149R, H149L, Q171L, Q171S, I173V, L178K, I179L, Q180M, F181L, K182D, D186T, G197S, D200K, S215P, K220N, V222I, W232Y, M336L, D341K, D341I, D341L, S349R, S349G, T368Y, D398I, V399R, Q560H, and any combination of two or more of the foregoing. Also provided are a kit including the polymerase, a polynucleotide encoding the polymerase, a method of synthesizing a polynucleotide using the polymerase, and a method of sequence a polynucleotide synthesized by the polynucleotide.

### CROSS-REFERENCE TO RELATED APPLICATIONS

This application claims benefit of priority from U.S. Provisional Patent Application No. 63/682,576, filed Aug. 13, 2024, the which is incorporated herein by reference in its entirety.

### SEQUENCE LISTING

The instant application contains an electronic sequence listing. The contents of the electronic sequence listing 6476.001AWO Sequence Listing XML.xml; Size: 20,758 bytes; and Date of Creation: Aug. 8, 2025, is herein incorporated by reference in its entirety.

### BACKGROUND

DNA polymerases are essential enzymes responsible for the synthesis and replication of DNA. In vivo, these enzymes facilitate DNA replication, repair, recombination, and genetic maintenance. In vitro, they serve as foundational tools for myriad molecular biology techniques, most notably the polymerase chain reaction (PCR). Thermostable DNA polymerase withstand repeated heating during thermal cycles without requiring replenishment.

Phi29 DNA polymerase, derived from the Bacillus subtilis phage Phi29, is a high-performance enzyme widely used in biotechnology for its strong strand displacement activity, high processivity, and proofreading capability. These properties make it indispensable in applications requiring isothermal DNA amplification, such as whole genome amplification (WGA), multiple displacement amplification (MDA), spatial genomics, single-cell genomics, and nucleic acid-based diagnostics.

A key feature of Phi29 DNA polymerase is its ability to synthesize long DNA fragments without dissociating from the template strand, enabling efficient amplification from limited DNA input. The enzyme's intrinsic 3′ to 5′ exonuclease activity provides high replication fidelity, which is critical in applications such as next-generation sequencing (NGS), cloning, and the creation of DNA libraries for downstream analyses.

In diagnostic contexts, Phi29 polymerase is employed in rolling circle amplification (RCA), whole genome amplification (WGA) for amplifying polynucleotides from a sample such as in advance of genome sequencing, and other isothermal techniques to sensitively detect pathogens, plasmids, or genetic alterations. Its ability to operate at a constant temperature eliminates the need for thermal cycling equipment, making it well-suited for field-deployable and point-of-care diagnostics.

Phi29 DNA polymerase also plays a role in the synthesis of fluorescently labeled probes for microarrays, FISH, and other nucleic acid detection platforms, further demonstrating its versatility across genomics and molecular biology.

Despite these advantages, the native enzyme has limitations, particularly in thermostability, expression yield, and compatibility with some manufacturing and diagnostic workflows. Accordingly, there is substantial interest in engineering Phi29 variants with improved thermostability, activity, expression, and manufacturability to enhance its performance and broaden its utility. The present disclosure is directed to overcoming these and other deficiencies in the art.

### SUMMARY

In an aspect, provided is a DNA polymerase, wherein the DNA polymerase has an amino acid sequence including at least 80% sequence identity with SEQ ID NO: 1, including one or more amino acid substitution wherein the one or more amino acid substitution is selected from the group consisting of A83S, N91I, R96H, R96C, M97V, G108D, G111V, R113H, V118S, S122N, L126I, P127L, A134T, D145V, D145N, D147V, H149R, H149L, Q171L, Q171S, I173V, L178K, I179L, Q180M, F181L, K182D, D186T, G197S, D200K, S215P, K220N, V222I, W232Y, M336L, D341K, D341I, D341L, S349R, S349G, T368Y, D398I, V399R, Q560H, and any combination of two or more of the foregoing, wherein the DNA polymerase exhibits polymerase activity. The one or more amino acid substitution may be selected from the group consisting of A83S, N91I, R96H, R96C, M97V, G108D, G111V, R113H, S122N, P127L, A134T, D145N, D145V, D147V, H149R, H149L, Q180M, D186T, G197S, D341L, T368Y, and any combination of two or more of the foregoing.

The DNA polymerase may further include one or more additional amino acid substitution, wherein the one or more additional amino acid substitution is selected from the group consisting of A83V, N91S, M97K, L107I, K110E, I15L, L123M, L123H, K131E, K138C, K138Q, 1172V, G191A, G197D, G197E, Y224K, F230Y, T231V, R236K, F237Y, E239G, I348V, T368M, T368F, Y369R, T372E, T373H, 1378K, K379R, A394G, and any combination of two or more of the foregoing. The one or more additional amino acid substitution may be selected from the group consisting of A83V, N91S, M97K, L107I, K110E, I115L, L123M, K131E, K138Q, G197D, G197E, Y224K, F237Y, T368F, K379R, A394G.

The amino acid sequence may include at least 85% sequence identity with SEQ ID NO: 1, at least 90% sequence identity with SEQ ID NO: 1, at least 95% sequence identity with SEQ ID NO: 1, at least 96% sequence identity with SEQ ID NO: 1, at least 97% sequence identity with SEQ ID NO: 1, at least 98% sequence identity with SEQ ID NO: 1, or at least 99% sequence identity with SEQ ID NO: 1

The amino acid sequence of the DNA polymerase may be selected from SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 14.

The DNA polymerase may exhibit one or more of higher yield, higher thermostability, higher solubility, and any combination of two or more of the foregoing relative to a DNA polymerase having the amino acid sequence of SEQ ID NO: 1.

In an aspect, provided is a polynucleotide encoding the DNA polymerase. In an aspect, provided is a vector including the polynucleotide. The vector may include a plasmid, cosmid, a bacterial artificial chromosome, or a phage vector.

In an aspect, provided is a kit, including the DNA polymerase, the polynucleotide encoding the polymerase, or the vector including the polynucleotide, and a reagent selected from the group consisting of a buffer, deoxyribonucleotides, and any two or more of the foregoing.

In an aspect, provided is a method of DNA amplification, including synthesizing polynucleotides complementary to a template strand by contacting the template strand with the DNA polymerase. The amplification may include multiple displacement amplification, whole genome amplification, plasmid amplification, viral amplification, rolling circle amplification, or preparation of a polynucleotide library for sequencing. In an aspect, provided is a method including sequencing one or more of the polynucleotides synthetized by the polynucleotide.

### BRIEF DESCRIPTION OF THE DRAWINGS

These and other features, aspects, and advantages of the present disclosure will become better understood when the following detailed description is read with reference to the accompanying drawings, wherein:

FIG. 1 shows examples of single amino acid substitutions relative to SEQ ID NO: 1 that resulted in an increased enrichment score, in accordance with aspects of the present disclosure.

FIG. 2 shows Multiple displacement amplification (MDA) reaction experiment testing activity of DNA polymerases having the amino acid sequences of SEQ ID NO: 1 (wild-type Phi29 DNA polymerase), SEQ ID NO: 2, and SEQ ID NO: 3, at 30° C. 34° C., 37° C. and 40° C. over the course of 75 minutes. Numerals signify the SEQ ID NO of the amino acid sequence of the DNA polymerase producing the depicted yield of double-stranded DNA (dsDNA). Top panels=linear scale, bottom panels=logarithmic scale.

FIG. 3 shows arrayed screening of examples of isolated DNA polymerases in accordance with aspects of the present disclosure. Rolling circle amplification (RCA) at different temperatures. DNA polymerases having the amino acid sequences of SEQ ID NO: 1 (wild-type Phi29 DNA polymerase, or WT), SEQ ID NO: 2, SEQ ID NO: 3, SEQ ID NO: 10, SEQ ID NO: 12, SEQ ID NO: 13, or SEQ ID NO: 14 at 37° C. and 42° C. over the course of 4 hours. Numerals signify the SEQ ID NO of the amino acid sequence of the DNA polymerase producing the depicted yield of double-stranded DNA (dsDNA). DNA polymerases having the amino acid sequences of SEQ ID NO's 2, 3, 10, 12, 13, and 14 made substantially more dsDNA than WT and continued producing dsDNA at the final measurement at 4 hours.

FIG. 4 shows rolling circle amplification (RCA) double-stranded DNA (dsDNA) amplification of DNA polymerases having amino acid sequences of SEQ ID NO: 1 (wild-type Phi29 DNA polymerase, or WT), SEQ ID NO: 2, SEQ ID NO: 4, SEQ ID NO: 5, SEQ ID NO: 6. SEQ ID NO: 7, SEQ ID NO: 8, SEQ ID NO: 9, SEQ ID NO: 10, and SEQ ID NO: 11 under 30° C. and 42° C. Numerals signify the SEQ ID NO of the amino acid sequence of the DNA polymerase producing the depicted yield of double-stranded DNA (dsDNA). DNA polymerases having the amino acid sequences of SEQ ID NO 2, 4, 5, 6, 7, 8, 9, 10, and 11 made substantially more dsDNA than WT and continue producing dsDNA through the final measurement at 4 hours.

FIG. 5 shows SDS page of uninduced (U) and induced (I) enzymes having the amino acid sequences of indicated SEQ ID NOs in strain B121 DE3. Arrows indicate higher solubility in cells induced to express DNA polymerase having the amino acid seq of SEQ ID NO 2 or 3 compared to that of SEQ ID NO: 1 (wild-type Phi29 DNA polymerase).

FIG. 6 shows multiple displacement amplification (MDA) reaction results for DNA polymerases having the amino acid sequences set out in SEQ ID NO 1 (wild-type Phi29 DNA polymerase), 2, and 3, with and without 30 min 30° C. preincubation. Numerals signify the SEQ ID NO of the amino acid sequence of the DNA polymerase producing the depicted yield of double-stranded DNA (dsDNA).

### DETAILED DESCRIPTION

This disclosure relates to a DNA polymerase with improved characteristics relative to naturally occurring, wild-type DNA polymerase and other commercially available polymerases. Particularly, disclosed herein are DNA polymerases sharing some amino acid sequence identity with Phi29 DNA polymerase, but where one or more amino acid substitutions have been made to a corresponding wild-type Phi29 amino acid sequence. DNA polymerases resulting from the amino acid substitutions to the corresponding wild-type Phi29 sequence as discloses herein possess superior qualities, capabilities, and characteristics, such as high yield, increased fragment length of the resulting polynucleotide product, high expressivity, high solubility, high overall stability, high thermal stability, low GC bias, high processivity, and high fidelity.

Unless stated otherwise, all scientific and technical terms used in this application are intended to have the meanings commonly understood by those skilled in the relevant field. The definitions provided herein are intended specifically for clarity in the context of this disclosure and should not be assumed to apply to other patents or applications, regardless of ownership. While alternative materials and methods may be used for implementing or evaluating the present invention, preferred examples are described in detail below. The definitions below are provided to facilitate understanding and interpretation of the invention.

As used throughout the specification and claims, the singular forms “a.” “an,” and “the” encompass the plural unless clearly indicated otherwise by the context. For example, “a protein” may refer to one or more proteins. “a cell” may refer to a population or mixture of cells, “an amino acid” may refer to one or more than one amino acid. “the nucleotide” may refer to one or more than one nucleotide, etc.

The term “about” as used herein in reference to a number following it means the phrase includes a range of from 10% below the number to 10% above the number. For example, “about 1 g” means “from 0.9 g to 1.1 g.”

The term “nucleic acid sequence” or “polynucleotide” refers to a linear arrangement of nucleotides in a polymer of DNA or RNA, or an analog thereof. A nucleic acid sequence may include deoxyribonucleotides, ribonucleotides, or chemically modified nucleotides, and may be single-stranded or double-stranded. The term encompasses naturally occurring sequences, as well as synthetic or recombinant sequences, including coding and non-coding regions, regulatory elements, primers, probes, or sequences encoding structural or functional domains of proteins. Nucleotides may be differentiated on the basis of their constituent nucleobase, such as adenine (A), guanine (G), cytosine (C), thymine (T), or uracil (U). A nucleotide including one of these nucleobases may be referred to generally according to its alphabetic referent (A, G, C, T, or U), even if it may possess a further chemical modification, such as constituents attached to a 3′, 5′, or other carbon of the ribose of the nucleotide, to 5′-phosphate of the nucleotide, or elsewhere.

The term “amino acid” or “any amino acid” as used here refers to any and all amino acids (i.e. organic molecules including an amino group and a carboxyl group, connected by a central carbon atom and including a side chain), including naturally occurring amino acids (e.g., α-amino acids, wherein the side chain is attached directly to the central carbon), unnatural amino acids, modified amino acids, and non-natural amino acids. It includes both D- and L-amino acids. Natural amino acids include those found in nature, such as, e.g., 23 aforementioned amino acids that combine into peptide chains to form the building-blocks of a vast array of proteins. These are primarily L stereoisomers, although a few D-amino acids occur in bacterial envelopes and some antibiotics. “Unnatural” or “non-natural” amino acids are non-proteinogenic amino acids (i.e., those not naturally encoded or found in the genetic code) that either occur naturally or are chemically synthesized. Over 140 unnatural amino acids are known and thousands of more combinations are possible. Examples of “unnatural” amino acids include β-amino acids (β3 and β2), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety, such as attached directly to the carboxyl or amino group or to the side chain, not naturally present on the amino acid and are included as examples where an amino acid is referred to herein.

One or more amino acid in an immunogenic polypeptide as disclosed herein may be an R-amino acid or an L-amino acid. One or more amino acid in an immunogenic polypeptide as disclosed herein may be a standard amino acid (i.e., selected from Alanine (Ala, or A), Arginine (Arg, or R), Asparagine (Asn, or N), Aspartic Acid (Asp, or D), Cysteine (Cys, or C), Glutamic acid (Glu, or E), Glutamine (Gln, or Q), Glycine (Gly, or G), Histidine (His, or H), Isoleucine (Ile, or I), Leucine (Leu, or L), Lysine (Lys, or K), Methionine (Met, or M), Phenylalanine (Phe, or F), Proline (Pro, or P), Serine (Ser, or S), Threonine (Thr. or T), Tryptophan (Trp, or W), Tyrosine (Tyr, or Y), Valine (Val, or V), Selenocysteine, N-formylmethionine, and Pyrrolysine).

An amino acid of one type of class may be substituted by another amino acid in the same class, or having similar chemical or physical properties, as would be understood by skilled persons, in what is referred to as a conservative substitution. A conservative substitution is one in which an amino acid is substituted for another amino acid that has similar properties, such that one skilled in the art of peptide chemistry would expect the secondary structure and hydropathic nature of the polypeptide to be substantially unchanged. In general, a substitution of one amino acid within the following groups for another amino acid within the following groups represents a conservative substitution. (1) Aliphatic amino acids Glycine (Gly, G), Alanine (Ala, A), Valine (Val, V), Leucine (Leu, L), Isoleucine (Ile, I); (2) hydroxyl or sulfur/selenium-containing Serine (Ser, S), Cysteine (Cys, C), Selenocysteine (Sec, U), Threonine (Thr, T), Methionine (Met, M); Cyclic Proline (Pro, P); Aromatic Phenylalanine (Phe, F), Tyrosine (Tyr, Y), Tryptophan (Trp, W); Basic Histidine (His, H), Lysine (Lys, K), Arginine (Arg, R); Acidic and their amides Aspartate (Asp, D), Glutamate (Glu, E), Asparagine (Asn, N), Glutamine (Gin, Q).

The term “amino acid sequence” means an uninterrupted sequence of amino acid residues in a polypeptide or protein. The sequence may be derived from a naturally occurring protein, a recombinant protein, or a synthetic peptide. Amino acid sequences may include standard or modified amino acids, and may be presented in either the single-letter or three-letter amino acid code. Variants, fusions, and fragments of proteins are also encompassed by this term unless otherwise specified.

“DNA polymerase” refers to an enzyme that catalyzes the synthesis of a DNA strand complementary to a DNA or RNA template by incorporating deoxyribonucleotide triphosphates (dNTPs). DNA polymerases may possess additional activities such as 3′ to 5′ exonuclease proofreading or strand displacement activity. The term encompasses wild-type enzymes, recombinant forms, engineered mutants, fusion constructs, and chemically modified variants, including enzymes derived from bacteriophages, bacteria, or other sources.

Unless otherwise specified, amino acid or nucleotide position numbers are reported relative to a reference sequence (e.g., wild-type Phi29 DNA polymerase). The position of a given residue in a variant sequence is identified based on sequence alignment with this reference, rather than by its absolute location in the variant sequence. The amino acid sequence for wild-type Phi29 DNA polymerase is provided as SEQ ID NO: 1.

As used herein, the term “recombinant” refers to a molecule-such as a nucleic acid or protein—that has been artificially modified by human intervention. This may include, for example, DNA generated through cloning, mutagenesis, directed evolution, or synthetic biology approaches. A “recombinant protein” may be produced by expressing a modified nucleic acid in a host organism, and may differ from its naturally occurring counterpart in sequence, source, or method of production.

The terms “DNA polymerase variant” or “DNA polymerase mutant” as used herein refers to a DNA polymerase that possesses one or more changes in its amino acid sequence compared to the wild-type Phi29-family DNA polymerase. Such variants typically share less than 100% sequence identity with the wild-type enzyme, and may exhibit at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity. These changes may include amino acid substitutions, insertions, or deletions introduced through protein engineering. Such variants or mutants, when aligned with the polypeptide sequence of SEQ ID NO: 1, have at least one amino acid that differs from that of a corresponding position in SEQ ID NO: 1.

For the purposes of this disclosure, unless expressly stated otherwise or context renders it unmistakably clear, the definite articles “a,” “an,” or “the” preceding an article, gerund, etc., includes examples having just one of the article, gerund, etc., or more than one of the article, gerund, etc.

The term “comprising” is used synonymously with the term “including,” meaning including but not limited to.

The phrase “comprising A, wherein A is selected from the group consisting of W, X, Y, Z, and any combination of two or more of the foregoing” means features in addition to the feature represented A, whether varieties in a category of which A is a member, or of a different category than A, may also be included. For example, “comprising a liquid, wherein the liquid is selected from the group consisting of water, ethanol, olive oil, and any combination of two or more of the foregoing” means including a liquid, wherein the liquid may be any one of, any two of, or all three of water, ethanol, and olive oil, and by itself the phrase does not by necessity exclude other features that may be non-liquids and does not by necessity exclude other liquids such as peanut oil, etc.

The combined conjunction “and/or” means any one or more or all of the features, qualities, materials, compositions, etc., listed in the series conjuncted by the combined conjunction. For example, “A, B, and/or C” includes all of: A alone, B alone, C alone, A and B without C. B and C without A, and A and B and C altogether.

An amino acid substitution may be a replacement of one or more amino acid in SEQ ID NO: 1 with a different amino acid at a corresponding location with a polypeptide aligned thereto. In an implementation, any one or more of amino acids 76 to 160 of the amino acid sequence represented by SEQ ID NO: 1 may be substituted with a different amino acid. For example, a DNA polymerase as disclosed herein may have 1 or more, 2 or more, 3 or more, 4 or more, 5 or more, 6 or more, 7 or more, 8 or more, 9 or more, 10 or more, 1I or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, or 63 or more amino acid substitutions relative to the amino acid sequence of SEQ ID NO: 1.

A DNA polymerase as disclosed herein may have from 1 to 63, from 1 to 62, from 1 to 61, from 1 to 60, from 1 to 59, from 1 to 58, from 1 to 57, from 1 to 56, from 1 to 55, from 1 to 54, from 1 to 53, from 1 to 52, from 1 to 51, from 1 to 50, from 1 to 49, from 1 to 48, from 1 to 47, from 1 to 46, from 1 to 45, from 1 to 44, from 1 to 43, from 1 to 42, from 1 to 41, from 1 to 40, from 1 to 39, from 1 to 38, from 1 to 37, from 1 to 36, from 1 to 35, from 1 to 34, from 1 to 33, from 1 to 32, from 1 to 31, from 1 to 30, from 1 to 29, from 1 to 28, from 1 to 27, from 1 to 26, from 1 to 25, from 1 to 24, from 1 to 23, from 1 to 22, from 1 to 21, from 1 to 20, from 1 to 19, from 1 to 18, from 1 to 17, from 1 to 16, from 1 to 15, from 1 to 14, from 1 to 13, from 1 to 12, from 1 to 11, from 1 to 10, from 1 to 9, from 1 to 8, from 1 to 7, from 1 to 6, from 1 to 5, from 1 to 4, from 1 to 3, or from 1 to 2 amino acid substitutions relative to the amino acid sequence of SEQ ID NO: 1. Any of the foregoing may include substitutions for one or more amino acid from amino acids 83 to 560 of SEQ ID NO: 1. Additionally and optionally, one or more amino acid of a DNA polymerase, which DNA polymerase may as disclosed herein have a degree of sequence identity with SEQ ID NO: 1, may correspond to a position that is N-terminal to amino acid 83 of SEQ ID NO: 1 and/or C-terminal to amino acid 560 of SEQ ID NO: 1.

A DNA polymerase as disclosed herein may have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 amino acid substitutions relative to the amino acid sequence of SEQ ID NO: 1. Any of the foregoing may include substitutions for one or more amino acid from amino acids 83 to 560 of SEQ ID NO: 1. Additionally and optionally, one or more amino acid of a DNA polymerase, which DNA polymerase may as disclosed herein have a degree of sequence identity with SEQ ID NO: 1, may correspond to a position that is N-terminal to amino acid 83 of SEQ ID NO: 1 and/or C-terminal to amino acid 560 of SEQ ID NO: 1.

Any one or more amino acid of SEQ ID NO: 1 may be substituted with any other amino acid. An amino acid substitution is identified by identifying the single-letter abbreviation for an amino acid followed by a numerical identification of its position in the polypeptide (with the N-terminal amino acid assuming the position of 1 and the C-terminal amino acid assuming the position of the highest numbered amino acid in the polypeptide. If a particular amino acid is identified for substitution at the position so identified, its one-letter abbreviation follows the numbered position. For example, L142 refers to a leucine amino acid at position 142 in the polypeptide and, depending on context, e.g. “L142 substitution,” indicates that an amino acid was substituted for the leucine at position 142. L142P indicates that a proline was substituted for leucine at position 142 in the polypeptide. A substitution may be a conservative substitution, or a non-conservative substitution.

Also disclosed herein is any polynucleotide (DNA or RNA) encoding any DNA polymerase disclosed herein. A person possessing ordinary skill in the art can envision a polynucleotide sequence encoding each and every DNA polymerase disclosed herein, including all variations in sequences of each and every given single DNA polymerase made possible because of codon degeneracy, whereby certain amino acids may be coded for by more than one triplet codon of nucleotides. See for example Snustad, D. P., & Simmons, M. J. (2003). Principles of Genetics (4th ed.). Every polynucleotide encoding a polypeptide having the sequence set out in SEQ ID NO: I and any variations thereof disclosed in Table 1, Table 2, or Table 3, below, whether possessing one or more amino acid substitution disclosed therein, in any combination, is explicitly included in the present disclosure. The polynucleotide may be included in a vector such as a plasmid or cosmid, transgenic organism such as a bacteria, an artificial chromosome, or a viral vector. Disclosed herein is a cell transfected or transducted with or expressing any of the foregoing polynucleotides, and a method of transfecting or transducting a cell with any of the foregoing polynucleotides, including by contacting the cell or an organism with any of the foregoing vectors or any of the foregoing polynucleotides.

“Storage buffer” refers to a buffered aqueous solution used to maintain the stability, solubility, and activity of DNA polymerase during storage. A storage buffer may contain one or more of the following: buffering agents (e.g., Tris-HCl), salts (e.g., KCl, NaCl), reducing agents (e.g., DTT), stabilizers (e.g., glycerol, detergents, proteins), or chelating agents (e.g., EDTA). The composition is formulated to minimize degradation or aggregation of the enzyme under refrigerated or frozen conditions.

“Reaction buffer” refers to a solution used to support the catalytic activity of DNA polymerase during DNA synthesis. It typically includes a buffering agent to maintain pH, salts to modulate ionic strength, and divalent cations (e.g., Mg²⁺ or Mn²⁺) required for enzyme activity. Reaction buffers may also include additives that enhance reaction efficiency, fidelity, or thermostability, and are optimized for specific applications such as amplification, sequencing, or isothermal reactions.

The amino acid sequence of wild-type Phi29 (also referred to as φ29) DNA polymerase is shown in SEQ ID NO: 1.

Table 1 presents amino acids that may, individually or in any combination of any two or more, be substituted for amino acid positions corresponding to those of Phi29 DNA polymerase, in accordance with aspects of the present disclosure, where numbers represent the corresponding position in SEQ ID NO: 1.

TABLE 1
Amino acid substitutions of SEQ ID NO: 1

| Amino acid of | |
|---|---|
| SEQ ID NO: 1 | Amino acid substitutions relative to SEQ ID NO: 1 |
| A83 | A83S |
| N91 | N91I |
| R96 | R96H, R96C |
| M97 | M97V |
| G108 | G108D |
| G111 | G111V |
| R113 | R113H |
| V118 | V118S |
| S122 | S122N |
| L126 | L126I |
| P127 | P127L |
| A134 | A134T |
| K135 | K135A, K135D, K135E, K135I, K135N, K135Q, K135R |
| D145 | D145V, D145N |
| D147 | D147V |
| H149 | H149R, H149L |
| Q171 | Q171S |
| I173 | I173V |
| L178 | L178K |
| I179 | I179L |
| Q180 | Q180M |
| F181 | F181L |
| K182 | K182D |
| D186 | D186T |
| G197 | G197S |
| D200 | D200K |
| S215 | S215P |
| K220 | K220N |
| V222 | V222I |
| W232 | W232Y |
| M336 | M336L |
| D341 | D341K, D341I, D341L |
| S349 | S349R, S349G |
| T368 | T368Y |
| D398 | D398I |
| V399 | V399R |
| Q560 | Q560H |

Table 2 presents a subset of the amino acids identified in Table 1 that may, individually or in any combination of any two or more, be substituted for amino acid positions corresponding to those of Phi29 DNA polymerase in accordance with aspects of the present disclosure, where numbers represent the corresponding position in SEQ ID NO: 1.

TABLE 2
Amino acid substitutions of SEQ ID NO: 1

| Amino acid of | Amino acid substitutions |
|---|---|
| SEQ ID NO: 1 | relative to SEQ ID NO: 1 |
| A83 | A83S |
| N91 | N91I |
| R96 | R96H, R96C |
| M97 | M97V |
| G108 | G108D |
| G111 | G111V |
| R113 | R113H |
| S122 | S122N |
| P127 | P127L |
| A134 | A134T |
| D145 | D145N, D145V |
| D147 | D147V |
| H149 | H149R, H149L |
| Q180 | Q180M |
| D186 | D186T |
| G197 | G197S |
| D341 | D341L |
| T368 | T368Y |

Table 3 presents amino acids that optionally may, individually or in any combination of any two or more, be substituted for amino acid positions corresponding to those of Phi29 DNA polymerase, in accordance with aspects of the present disclosure, where numbers represent the corresponding position in SEQ ID NO: 1, in addition to one or more amino acid substitutions identified in Table 1 or Table 2.

TABLE 3
Additional amino acid substitutions of SEQ ID NO: 1

| Amino acid of | Amino acid substitutions |
|---|---|
| SEQ ID NO: 1 | relative to SEQ ID NO: 1 |
| A83 | A83V |
| N91 | N91S |
| M97 | M97K |
| L107 | L107I |
| K110 | K110E |
| I115 | I115L |
| L123 | L123M, L123H |
| K131 | K131E |
| K138 | K138C, K138Q |
| I172 | I172V |
| G191 | G191A |
| G197 | G197D, G197E |
| Y224 | Y224K |
| F230 | F230Y |
| T231 | T231V |
| R236 | R236K |
| F237 | F237Y |
| E239 | E239G |
| I348 | I348V |
| T368 | T368M, T368F |
| Y369 | Y369R |
| T372 | T372E |
| T373 | T373H |
| I378 | I378K |
| K379 | K379R |
| A394 | A394G |

Table 4 presents a subset of the amino acids identified in Table 3 that optionally may, individually or in any combination of any two or more, be substituted for amino acid positions corresponding to those of Phi29 DNA polymerase, in accordance with aspects of the present disclosure, where numbers represent the corresponding position in SEQ ID NO: 1, in addition to one or more amino acid substitutions identified in Table 1 or Table 2.

TABLE 4
Additional amino acid substitutions of SEQ ID NO: 1

| Amino acid of | Amino acid substitutions |
|---|---|
| SEQ ID NO: 1 | relative to SEQ ID NO: 1 |
| A83 | A83V |
| N91 | N91S |
| M97 | M97K |
| L107 | L107I |
| K110 | K110E |
| I115 | I115L |
| L123 | L123M |
| K131 | K131E |
| K138 | K138Q |
| G197 | G197D, G197E |
| Y224 | Y224K |
| F237 | F237Y |
| T368 | T368F |
| K379 | K379R |
| A394 | A394G |

Table 5 identifies SEQ ID NOs of DNA polymerases disclosed herein whose sequences correspond to that of SEQ ID NO: 1 except for the combinations, or stacking, of amino acid substitutions each has, relative to the corresponding amino acid of SEQ ID NO: 1.

TABLE 5
SEQ ID NOs and stacked amino acid substitutions
for DNA polymerases disclosed herein

| SEQ ID | |
|---|---|
| NO: | Amino acid substitutions relative to SEQ ID NO: 1 |
| 2 | L107I, L178K, I179L, Q180M, K182D, Y224K, R236K, M336L, D341K, |
| | I348V, S349R, T368Y, T373H, I378K, A394G |
| 3 | L107I, R113H, L178K, I179L, Q180M, K182D, Y224K, R236K, M336L, |
| | D341K, I348V, S349R, T368Y, T373H, I378K, A394G |
| 4 | L107I, V118L, H149N, L178K, I179L, Q180M, K182D, Y224K, R236K, |
| | M336L, D341K, I348V, S349R, T368Y, T373H, I378K, A394G |
| 5 | L107I, D14SN, H149L, L178K, I179L, Q180M, K182D, Y224K, R236K, |
| | M336L, D341K, I348V, S349R, T368Y, T373H, I378K, A394G |
| 6 | L107I, R113H, V118S, D145N, I173V, L178K, I179L, Q180M, K182D, |
| | Y224K, R236K, M336L, D341K, I348V, S349R, T368Y, T373H, I378K, |
| | A394G |
| 7 | L107I, Q171L, L178K, I179L, Q180M, K182D, Y224K, R236K, M336L, |
| | D341K, I348V, S349R, T368Y, T373H, I378K, A394G |
| 8 | L107I, V118S, Q171L, L178K, I179L, Q180M, K182D, D186T, Y224K, |
| | R236K, M336L, D341K, I348V, S349R, T368Y, T373H, I378K, A394G |
| 9 | L107I, I173V, L178K, I179L, Q180M, K182D, Y224K, R236K, M336L, |
| | D341K, I348V, S349R, T368Y, T373H, I378K, A394G |
| 10 | L107I, H149L, L178K, I179L, Q180M, K182D, D186T, Y224K, R236K, |
| | M336L, D341K, I348V, S349R, T368Y, T373H, I378K, A394G |
| 11 | L107I, D145V, L178K, I179L, Q180M, K182D, D186T, Y224K, R236K, |
| | M336L, D341K, I348V, S349R, T368Y, T373H, I378K, A394G |
| 12 | L107I, R113H, H149L, Q171L, I173V, L178K, I179L, Q180M, K182D, |
| | Y224K, R236K, M336L, D341K, I348V, S349R, T368Y, T373H, I378K, |
| | A394G |
| 13 | L107I, V118S, D145N, L178K, I179L, Q180M, F181L, K182D, Y224K, |
| | R236K, M336L, D341K, I348V, S349R, T368Y, T373H, I378K, A394G |
| 14 | L107I, V118S, L178K, I179L, Q180M, F181L, K182D, Y224K, R236K, |
| | M336L, D34IK, I348V, S349R, T368Y, T373H, I378K, A394G |

Disclosed herein are DNA polymerases having any one or more of the amino acid substitutions presented in Tables 1 or 2, optionally also including one or more amino acid substitution presented in Table 3 or 4, such as, without limitation, DNA polynucleotides disclosed in Table 5 and Table 6 (in the Examples). All possible combinations or permutations of an amino acid substitution for one of the corresponding amino acids of SEQ ID NO: 1 with any one or more of any of the other amino acid substitutions for another one or more of the corresponding amino acids of SEQ ID NO: 1 as set out in Table 1 or 2, optionally also including one or more of the amino acid substitutions as set out in Table 3 or 4, including as non-limiting implementations as set out in Tables 5 and 6 (see below), is explicitly contemplated, hereby disclosed, and explicitly included in a DNA polymerase accordance with the present disclosure. For the avoidance of doubt, phrases such as “one or more of X₁, X₂, X₃, X₄, . . . Xₙ, and any combination thereof” and the like, wherein each X represents an amino acid substitution identified in any one of Tables 1, 2, 3, or 4, includes, without limitation unless explicitly stated so, a protein with any one of the identified amino acid substitutions identified in the series, and two of the amino acid substitutions identified in the series, any three of the amino acid substitutions identified in the series, etc., up to and including all n of the amino acid substitutions identified in the series.

A polymerase is an enzyme that catalyzes the synthesis of a nucleic acid strand complementary to a template strand by sequentially adding nucleotides to the 3′-end of a growing nucleic acid chain. In the context of DNA replication or amplification, a DNA polymerase refers specifically to an enzyme that facilitates the incorporation of deoxyribonucleotide triphosphates (dNTPs) into a newly forming DNA strand using a DNA template, in a 5′ to 3′ direction. A polypeptide possesses “polymerase activity,” as the term is used herein, if it exhibits an enzymatic function of synthesizing nascent DNA in a template-dependent manner.

In certain implementations, a DNA polymerase may possess strand displacement activity, which enables the enzyme to displace downstream DNA strands while synthesizing new DNA without the need for a separate helicase. Strand displacement polymerases are particularly useful in isothermal amplification techniques, where continuous synthesis and displacement allow exponential amplification without thermal cycling.

A DNA polymerase suitable for use in nucleic acid amplification reactions is wild-type Phi29 DNA polymerase, having the amino acid sequence of SEQ ID NO: 1, as are the other DNA polymerases disclosed herein, including as disclosed in Table 1, Table 2, Table 3, Table 4, Table 5, and Table 6 (see below). DNA polymerases as disclosed herein exhibit robust polymerase activity with high fidelity due to 3′ to 5′ exonuclease proofreading function, and are capable of synthesizing long DNA fragments under isothermal conditions. The DNA polymerases disclosed herein support multiple displacement amplification (MDA), a method for whole genome amplification that allows for uniform and unbiased amplification of genomic DNA. Following is a description of non-limiting implementations of methods of using a DNA polymerase, including a Phi29 DNA polymerase or other DNA polymerase as disclosed herein, including a DNA polymerase having amino acid sequence identity with Phi29 DNA polymerase but including one or more amino acid substitution as discloses herein, such as identified in the present paragraph.

As used in the present application, polymerase activity includes the ability of a DNA polymerase to catalyze the formation of phosphodiester bonds between nucleotides in a newly synthesized DNA strand using a pre-existing strand of DNA as a template, optionally with strand displacement and proofreading capabilities. Polymerase activity may be exhibited by wild-type DNA polymerases having the amino acid sequence of SEQ ID NO: 1, as well as the DNA polymerases disclosed herein, including those of Table 1, Table 2, Table 3, Table 4. Table 5, and Table 6 (see below).

In some implementations, a DNA polymerase as disclosed herein may include one or more chemical constituent covalently or non-covalently attached or conjugated thereto, such as to a side chain of an amino acid, or to its N- or C-terminus. A DNA polymerase as disclosed herein may be attached to a substrate, such as via such a linker or other molecular or other connection. Unless otherwise stated herein or made clear by context, all DNA polymerases as disclosed herein may include such an additional chemical constituent, provided the amino acid sequence is disclosed herein is otherwise the same. For example, a cysteine amino acid is still referred to herein as a cysteine amino acid even though its side chain may be conjugated to another molecule through a disulfide or other linkage, a lysine amino acid is still referred to herein as a lysine amino acid even though its side chain may be conjugated to another molecule through an acyl- or other linkage, or any other amino acid referred to herein is still referred to as the same amino acid even though its side chain or amino or carboxyl group may be conjugated to another molecule.

Non-limiting implementations of uses of any and all of the DNA polymerases disclosed herein, without limitation, include whole genome amplification (WGA), multiple displacement amplification (MDA), and rolling circle amplification (RCA).

WGA is a molecular biology technique used to amplify the entire genomic content of a DNA sample, thereby generating sufficient quantities of DNA for downstream analysis when starting material is limited. This approach is particularly valuable for applications involving small or degraded samples, such as single-cell genomics, preimplantation genetic diagnosis, forensic investigations, and metagenomic studies. Central to the WGA process is the use of DNA polymerases, which catalyze the synthesis of new DNA strands based on existing templates. Any of the DNA polymerases disclosed herein are hereby disclosed for use in WGA.

WGA may be performed using isothermal or thermocycling amplification methods, both of which use on DNA polymerases with enzymatic properties appropriate for the method. One widely used isothermal WGA method is multiple displacement amplification (MDA), which conventionally employs use of DNA polymerases, such as wild-type the Phi29 DNA polymerase having the amino acid sequence set out in SEQ ID NO: 1. All of the DNA polynucleotides disclosed herein, including without limitation those disclosed in Table 1, Table 2, Table 3, Table 4, Table 5, and Table 6, are explicitly disclosed for use in WGA. Phi29 DNA polymerase is a high-fidelity, strand-displacing enzyme capable of synthesizing long DNA fragments at a constant temperature, typically around 30° C. In this approach, the genomic DNA is first denatured and annealed with random hexamer primers. Phi29 DNA polymerase then extends these primers, continuously synthesizing new strands while displacing downstream DNA, resulting in a hyperbranched, high-yield amplification of the entire genome. The enzyme's strong strand-displacement activity, proofreading capability, and processivity make it particularly well-suited for generating unbiased and representative amplification products. As disclosed herein, the DNA polymerases disclosed herein, including those disclosed in Table 1, Table 2, Table 3, Table 4, Table 5, and Table 6, without limitation, possess advantageously improved characteristics over Phi29 DNA polymerase having the amino acid sequence of SEQ ID NO: 1 and may therefore be preferred DNA polymerases for use in WGA over wild-type Phi29 of SEQ ID NO: 1.

DNA polymerases play a critical role in WGA by enabling the synthesis of new DNA strands complementary to the template DNA. The choice of DNA polymerase determines how an amplification method may be performed and its strengths and benefits as well as its potential weaknesses and shortcomings, such as may be related to one or more of yield, activity, stability, solubility, and any combination of two or more of the foregoing.

The amplified DNA generated through WGA can be used in a variety of downstream applications, including sequencing (including next generation sequencing, nanopore sequencing, etc.), single nucleotide polymorphism (SNP) analysis, copy number variation detection, quantitative PCR, and microarray-based assays. Phi29 DNA polymerases and variants thereof, including DNA polymerases as disclosed herein, may find uses in processes such as amplifying DNA from small amounts of starting material, such as via RCA. MDA, or WGA, processes useful in, for example, preparing DNA templates for sequencing. Phi29 DNA polymerase and variants thereof, including DNA polymerases as disclosed herein, may also be used in nanopore-based sequencing methods, where synthesized nascent DA strands may be fed through nanopores in a membrane for sequencing. Phi29 DNA polymerase and variants thereof, including DNA polymerases as disclosed herein, may also be used Single Molecule Real-Time (SMRT) sequencing.

In plasmid sequencing preparation, use of a DNA polymerase as disclosed herein in an RCA method may allow for the rapid amplification of plasmid DNA from minute amounts of bacterial culture or even single colonies, obviating the need for lengthy growth and traditional DNA isolation procedures. This approach is particularly valuable for situations with limited starting material, such as sequencing plasmids from challenging-to-culture organisms or those available in low copy numbers.

The ability of DNA polymerases as disclosed herein to amplify DNA from minute amounts makes it ideal for single-cell sequencing applications. MDA using a DNA polymerase as disclosed herein enables whole-genome amplification from single cells, providing sufficient DNA for downstream sequencing and analysis.

Amplification methods using a DNA polymerase as disclosed herein, such as MDA, are also useful in spatial genomics, where DNA is amplified directly from tissues or cells while preserving their spatial organization. This allows researchers to investigate genetic variations and gene expression profiles within a tissue context, gaining insights into disease progression or tissue development and/or screening for drugs.

A DNA polymerase as disclosed herein may be provided as a stand-alone product, such as in an aqueous solution, or as a dried or lyophilized composition. It may also be included, in either of the foregoing states or otherwise, in a kit, such as for use in a method involving DNA polymerization. A kit may include one or more DNA polymerase disclosed herein. A kit may include a polynucleotide encoding any one or more DNA polymerase disclosed herein. A kit may include one or more vector including a polynucleotide encoding any DNA polymerase disclosed herein, such as a plasmid, cosmid, transgenic organism such as a bacteria, an artificial chromosome, or a viral vector. A kit including DNA polymerase or polynucleotide encoding a DNA polymerase as disclosed herein may include, in addition to the enzyme itself, various reagents, buffers, and materials necessary or advantageous for the performance of whole genome amplification (WGA) or other DNA amplification reactions. The kit may include a purified preparation of the DNA polymerase, optionally provided in a storage buffer containing glycerol or other stabilizing agents to preserve enzymatic activity during storage and use.

In some implementations, a kit may include a reaction buffer formulated to optimize the activity of the DNA polymerase. The reaction buffer may contain one or more buffering agents (e.g., Tris-HCl), divalent metal ions (e.g., MgCl₂ or MnCl₂) as cofactors for DNA polymerase activity, and/or salts (e.g., NaCl or KCl) to maintain appropriate ionic strength. A buffer may optionally include components that enhance strand displacement or reduce nonspecific priming, such as nonionic detergents (e.g., Tween-20 or Triton X-100), bovine serum albumin (BSA), or other protein stabilizers.

A kit may further include primers suitable for initiating DNA synthesis. In certain implementations, primers may be random primers, such as random hexamers, which anneal nonspecifically across the genomic DNA to allow uniform initiation of amplification. The primers may be provided in a separate tube or vial or pre-mixed with the reaction buffer.

In certain implementations, a kit may include a deoxynucleotide triphosphate (dNTP) mix containing the four standard nucleotides (dATP, dTTP, dCTP, and dGTP) at concentrations sufficient to support extensive DNA synthesis by the DNA polymerase. A dNTP mix may be provided as a separate reagent or in combination with other reaction components.

A kit may also include reagents for template preparation, such as lysis buffer or denaturation buffer, to facilitate the release of genomic DNA from cells or other biological samples prior to amplification. Where appropriate, the kit may include components for use in a DNA denaturation step, such as an alkaline treatment solution (e.g., NaOH), or heat-denaturation protocol, along with a corresponding neutralization buffer.

A kit may further include control DNA templates, such as a known genomic DNA sample or synthetic DNA, to validate the amplification process. In certain embodiments, the kit may also include a protocol or instruction manual outlining recommended conditions for whole genome amplification, including suggested reagent volumes, incubation temperatures, and reaction durations.

A kit may include one or more auxiliary components, such as nuclease-free water, reaction tubes, or enzyme-compatible plasticware, to facilitate convenient and contamination-free reaction setup.

### EXAMPLES

The following examples are intended to illustrate particular embodiments of the present disclosure, but are by no means intended to limit the scope thereof.

### Example 1. Pooled Screen to Select for Phi29 Variants with Improved Activity at High Temperatures

### Methods

To identify amino acid substitutions that enable a DNA polymerase as disclosed herein to produce more specific dsDNA at high temperatures, an ultra-high-throughput droplet-based screening platform was developed based on a compartmentalized self-replication (CSR) strategy (see F. J. Ghadessy et al., Directed evolution of polymerase function by compartmentalized self-replication, Proc. Natl. Acad. Sci. U.S.A. 98 (8) 4552-4557) with modifications. In this platform, a library of DNA polymerases similar to wild-type Phi29 DNA polymerase (SEQ ID NO: 1), which DNA polymerase is sometimes referred to herein simply as “Phi29” or “Phi 29” or “<φ29” or “<φ 29,” but with one or more amino acid substitution is cloned as a pool into E. coli, grown, induced with IPTG, and each cell captured in a droplet. A Phi29 DNA polymerase with one or more amino acid substitution (relative to SEQ ID NO: 1) as disclosed herein is sometimes referred to herein as a Phi29 “mutant” or “variant” enzymes or the amino acid substitutions for amino acids of DNA polymerases as disclosed herein relative to the amino acid sequence of SEQ ID NO: 1 are sometimes referred to herein as “mutations” or “substitutions” or the like. Each sample contains billions of droplets, with each droplet holding either zero or one (ideally) or more cells expressing a designed enzyme. Each droplet also contains all reagents required for RCA. Under selective pressure, high-performing enzymes amplify their own DNA more efficiently. Less active enzymes amplify their DNA to a lesser extent. The RCA reaction is allowed to run, then the same is heat inactivated, droplets broken, and newly produced dsDNA extracted and sequenced via short-read next-generation sequencing. To determine the effect of each mutation, we first measured the relative enrichment or depletion of each genotype using the read counts for each genotype before and after iCSR, averaging across multiple biological replicates and RCA temperatures. Ridge regression was then used to estimate a coefficient (weight) for each substitution that best explains the data. Beneficial substitutions receive positive weights, while deleterious ones receive negative weights. These weights were then used to choose combinations of substitutions for subsequent stacking and arrayed screening.

Certain individual amino acid substitutions were identified for consideration from possibilities generated by using open source mutation scoring tools, including ESM, ProteinMPNN, EVcouplings, and RaSP. From these, rational design was employed to identify and select amino acid substitutions for high-throughput CSR and functional analysis. Enzyme-DNA interface residues, active sites, surface residues, and structurally supporting residues were rationally identified from DNA polymerase crystal structures (including PDB:2pyl and PDB:1xhx) and for generating candidate targets. Mutations were scored and filtered by combining multiple methods designed for identifying improved polymerase attributes, as disclosed herein. Mutations were combined and rationally reviewed to confirm absence of structural conflicts created by candidate amino acid substitutions (for example, including but not limited to breaking hydrophobic core, conserved regions, active sites, DNA binding, hydrogen bonds or causing steric collisions). Enzymes were synthesized and screened as disclosed herein, including multiple dimension screening data derived from DNA polymerase enzymes designed for multiple, simultaneous enhanced properties.

### Construction of Mutant Library

Mutant oligo pools were cloned into a wild-type or mutant Phi29 CDS already cloned into an expression vector, and transformed into Escherichia coli BL21(DE3) cells. Cells were plated, and resulting colonies were scraped and pooled to generate glycerol stocks.

### Culture and Induction

One glycerol stock (I mL) of pooled library cells was inoculated into 99 mL LB medium containing carbenicillin (100 μg/mL) and grown overnight at 37° C. with shaking at 300 rpm. Following overnight growth, 10 mL fresh LB containing carbenicillin (100 μg/mL) was inoculated with the overnight culture at a starting OD600=0.125. The culture was grown at 37° C. to OD600=1.0, induced with IPTG (1 mM final concentration), and incubated for an additional 3 hours at 30° C. Cells were then pelleted, aliquoted, and stored at −80° C. until used.

### Preparation of Emulsions

An aliquot of IPTG-induced E. coli cells was resuspended in I mL of 1× Tango buffer (33 mM Tris-acetate pH 7.9, 10 mM Mg-acetate, 66 mM K-acetate, 0.1 mg/mL BSA) containing freshly prepared lysozyme (0.5 mg/mL final concentration). After a 5-minute incubation at room temperature, cells were pelleted, washed, and resuspended in 300 μL CSR mix (1× Tango buffer, 50 μM exonuclease-resistant random primer (5′-NpNpNpNpNp′N-3′) mix, 0.5 μM each of tailed plasmid-specific primers, 1 mM dNTPs). The cells in the CSR mix were slowly pipetted to ensure homogeneity.

The aqueous phase (CSR mix with cells) was added dropwise over 2 minutes into 700 μL of the oil phase (mineral oil containing 2% v/v ABIL EM 90 and 0.055% v/v Triton X-100), while continuously vortexing. The emulsion was vortexed for an additional 5 minutes and homogenized using a BeadBug 6 homogenizer at 4350 rpm for 10 minutes in a cold room. The prepared emulsions were visually inspected to confirm homogeneity.

Cell Lysis and iCSR Amplification

Emulsions were subjected to five freeze-thaw cycles, consisting of 30 minutes freezing at −80° C. followed by 5 minutes thawing at the desired selection temperature. Following cell lysis, emulsions were incubated at the selection temperature for 4 hours for isothermal amplification by Phi29 DNA polymerase variants expressed within the emulsified compartments. Reactions were terminated by heating emulsions to 80° C. for 15 minutes.

### DNA Recovery and Amplification

The aqueous phase containing amplified DNA was recovered from emulsions via phase extraction using water-saturated diethyl ether and ethyl acetate washes, followed by phenol-chloroform extraction and ethanol precipitation. Purified DNA was digested with restriction endonucleases (DpnI and AlwNI) to remove residual plasmid template and linearize the amplified product. The selected Phi29 DNA polymerase gene fragments were PCR-amplified using primers flanking the polymerase gene region and sent for Illumina sequencing, along with the input population for read count normalization.

### Model Fitting

To quantify and model the enrichment of mutants, the log 2 ratio of output reads (post-selection) to input reads (pre-selection) was calculated, resulting in an experimental enrichment score for each genotype. Each mutation within the sequenced genotypes was one-hot encoded. Ridge regression, combined with cross-validation, was then applied to these one-hot encoded genotypes to predict the experimentally derived enrichment scores. This statistical modeling yielded weights and coefficients indicating the impact of each substitution on enzyme performance.

It is important to note that even beneficial mutations can have negative weights and enrichment scores, as these values depend on the combined fitness of all genotypes; mutants can be better than wild-type but still depleted because they are in a pool together with far superior genotypes.

### Hit Stacking

Top-performing amino acid substitutions identified through model fitting were incorporated into either the wild-type Phi29 DNA polymerase or a previously engineered Phi29 variant (that is, a DNA polymerase already including on or more amino acid substitution relative to SEQ ID NO: 1). These stacked mutant constructs were individually expressed and evaluated using arrayed screening methods. Arrayed screening involved the systematic testing of each stacked mutant variant under controlled conditions, validating and quantifying improvements in thermostability, catalytic rate, and overall activity relative to the parent enzymes.

### Iterate (Design, Screen, and Hit-Stack the Next Library)

Based on insights from model fitting and hit stacking, the iterative design of subsequent mutant libraries was carried out. New combinations of beneficial mutations were designed, introduced into a given DNA polymerase gene, and subjected to another round of pooled screening and hit stacking. This cyclical process of design, screening, and validation via pooled and arrayed screening allowed continuous improvement in DNA polymerase structure.

### Results

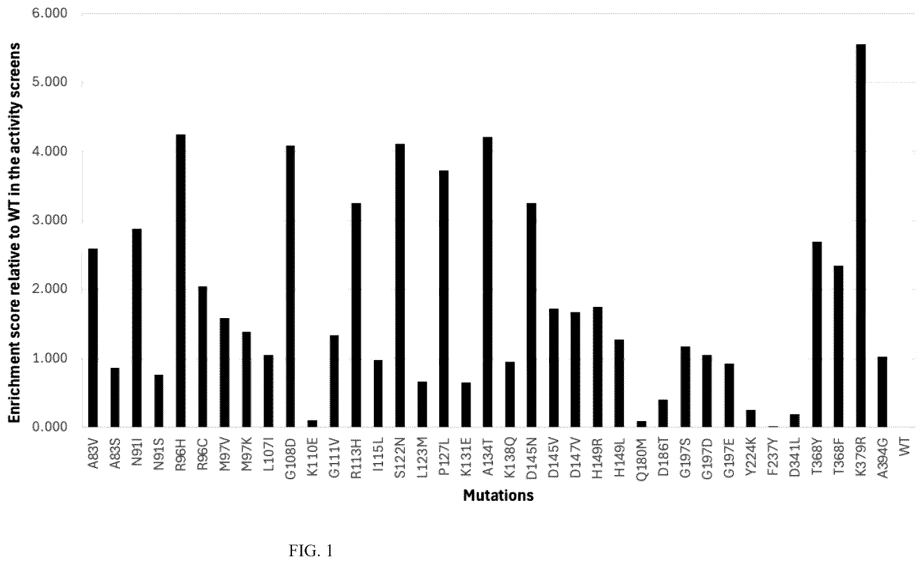

The following, non-limiting implementations of the present disclosure include single mutations with improved activity in screening relative to the DNA polymerase having the amino acid sequence of SEQ ID NO: 1: A83V, A83S, N91I, N91S, R96H, R96C, M97V, M97K, L107I, G108D, K110E, G111V, R113H, I115L, S122N, L123M, P127L, K131E, A134T, K138Q, D145N, D145V, D147V, H149R, H149L, Q180M, D186T, G197S, G197D, G197E, Y224K, F237Y, D341L, T368Y, T368F, K379R, A394G. See FIG. 1.

Additional amino acid substitutions (single or stacked) showing improved activity in screening relative to the DNA polymerase having the amino acid sequence of SEQ ID NO: 1 are listed in in Table 6, where an enrichment score greater than 1 indicates improved activity in screening relative to the DNA polymerase having the amino acid sequence of SEQ ID NO: 1 (that is, relative to wild-type Phi29 DNA polymerase).

TABLE 6
Beneficial mutation combinations. (that only use mutations from provisional
singles) showed higher activity than WT in our screening experiments.

| | Enrichment |
|---|---|
| Amino Acid Substitution(s) relative to SEQ ID NO: 1 | score |
| L178K G197E D200K | 3.57 |
| G197E D200K R236K E239G | 3.36 |
| L178K G197D Y224K R236K E239G | 3.27 |
| G197E D200K | 3.13 |
| L178K D186T G197E | 3.07 |
| L178K K182D D200K Y224K R236K E239G | 3.00 |
| G197E D200K S215P E239G | 2.91 |
| G197E D200K Y224K T231V W232Y E239G | 2.90 |
| Q171L I172V I173V L178K I179L Q180M K182D G191A G197S | 2.80 |
| Y224K T231V W232Y E239G | |
| Q180M G191A G197E S215P Y224K F230Y T231V W232Y R236K | 2.75 |
| E239G | |
| G197E V222I Y224K E239G | 2.69 |
| I172V L178K G197S Y224K T231V W232Y R236K E239G | 2.68 |
| G191A G197S S215P Y224K T231V W232Y R236K E239G | 2.68 |
| G197E V222I R236K E239G | 2.68 |
| G191A G197S S215P Y224K | 2.67 |
| G197S R236K E239G | 2.66 |
| G197S Y224K E239G | 2.65 |
| G191A G197S Y224K | 2.64 |
| D186T G197E Y224K | 2.63 |
| D186T G197E V222I R236K E239G | 2.60 |
| G197E V222I Y224K T231V W232Y R236K E239G | 2.58 |
| L178K G191A G197S S215P T231V | 2.57 |
| Q180M V222I Y224K E239G | 2.53 |
| L178K K182D D200K S215P E239G | 2.50 |
| G197E Y224K R236K | 2.50 |
| L178K K182D G197S S215P R236K E239G | 2.49 |
| G191A G197S D200K Y224K | 2.46 |
| M336L T368Y | 2.44 |
| L178K K182D V222I Y224K E239G | 2.43 |
| G191A G197S V222I Y224K T231V W232Y R236K E239G | 2.43 |
| G191A G197S Y224K E239G | 2.41 |
| G197D V222I Y224K R236K E239G | 2.40 |
| G197S S215P V222I Y224K T231V W232Y R236K E239G | 2.39 |
| L178K D200K S215P R236K E239G | 2.39 |
| D186T G197E R236K E239G | 2.38 |
| I179L G197D Y224K T231V W232Y R236K E239G | 2.38 |
| L178K G191A G197S S215P Y224K T231V W232Y E239G | 2.36 |
| G191A G197E Y224K E239G | 2.35 |
| G191A G197S E239G | 2.32 |
| D186T G197S S215P Y224K R236K E239G | 2.32 |
| L178K G197D E239G | 2.31 |
| L178K K182D Y224K R236K | 2.31 |
| D186T V222I Y224K E239G | 2.29 |
| G197E Y224K W232Y E239G | 2.29 |
| L178K G197D Y224K F230Y T231V W232Y R236K E239G | 2.28 |
| L178K G197D V222I W232Y E239G | 2.28 |
| D186T Y224K R236K | 2.26 |
| G197S S215P V222I Y224K F230Y T231V W232Y R236K F237Y | 2.26 |
| E239G | |
| G197D S215P | 2.26 |
| I172V G197S S215P V222I Y224K T231V W232Y R236K E239G | 2.26 |
| I172V G191A G197S Y224K T231V W232Y E239G | 2.25 |
| L178K D200K Y224K R236K E239G | 2.22 |
| G197E Y224K T231V W232Y E239G | 2.21 |
| T372E A394G | 2.20 |
| G197S V222I Y224K T231V W232Y R236K E239G | 2.19 |
| Q171S G191A G197S Y224K T231V W232Y E239G | 2.18 |
| G197S V222I Y224K E239G | 2.15 |
| L178K G197S | 2.14 |
| D186T G197E T231V R236K E239G | 2.14 |
| L178K V222I Y224K R236K E239G | 2.13 |
| L178K I179L Q180M K182D Y224K W232Y E239G | 2.13 |
| G197S Y224K R236K E239G | 2.13 |
| L178K K182D G197E E239G | 2.13 |
| T368F A394G | 2.11 |
| D186T G197E | 2.11 |
| G197E S215P V222I Y224K F230Y T231V W232Y R236K F237Y | 2.10 |
| E239G | |
| G197E Y224K E239G | 2.10 |
| L178K D200K Y224K T231V R236K | 2.10 |
| L178K Y224K T231V R236K E239G | 2.10 |
| G197D S215P E239G | 2.09 |
| G197E D200K W232Y E239G | 2.08 |
| G197D Y224K | 2.08 |
| T368F T372E | 2.07 |
| D186T G197E S215P V222I Y224K T231V W232Y R236K E239G | 2.07 |
| G197D V222I W232Y E239G | 2.07 |
| M336L A394G | 2.06 |
| M336L S349G | 2.06 |
| L178K G197D | 2.06 |
| G197E Y224K | 2.05 |
| D200K Y224K T231V R236K | 2.04 |
| G197E R236K E239G | 2.03 |
| Q180M G191A V222I Y224K | 2.02 |
| L178K G191A S215P Y224K E239G | 2.02 |
| D186T G197E S215P V222I Y224K F230Y T231V W232Y R236K | 2.02 |
| E239G | |
| L178K Q180M E239G | 2.01 |
| L178K G197D Y224K W232Y E239G | 2.01 |
| Q180M G191A G197E S215P Y224K T231V W232Y E239G | 2.01 |
| L178K S215P V222I Y224K T231V R236K E239G | 2.00 |
| G197E S215P V222I E239G | 2.00 |
| L178K G191A G197E S215P Y224K | 2.00 |
| I172V G197S | 2.00 |
| Q180M D200K S215P Y224K T231V E239G | 1.99 |
| L178K G191A Y224K E239G | 1.99 |
| G191A G197S | 1.98 |
| L178K K182D S215P V222I Y224K F230Y T231V W232Y R236K | 1.96 |
| F237Y E239G | |
| D200K S215P Y224K R236K | 1.95 |
| G197S W232Y E239G | 1.95 |
| K379R A394G | 1.94 |
| G197E Y224K T231V W232Y R236K E239G | 1.93 |
| Q180M G197D E239G | 1.93 |
| D200K Y224K R236K E239G | 1.93 |
| L178K S215P V222I Y224K T231V W232Y R236K F237Y E239G | 1.92 |
| L178K K182D G197E | 1.91 |
| T368Y T373H I378K | 1.91 |
| G197E S215P V222I Y224K F230Y T231V W232Y R236K E239G | 1.90 |
| G197D E239G | 1.89 |
| G197D T231V R236K | 1.88 |
| G191A G197S T231V E239G | 1.87 |
| G191A G197S R236K | 1.87 |
| L178K G191A G197S Y224K T231V W232Y E239G | 1.85 |
| G191A G197S Y224K T231V W232Y E239G | 1.85 |
| K182D G191A G197S | 1.83 |
| I172V Q180M R236K E239G | 1.83 |
| I172V Q180M G191A E239G | 1.82 |
| Q180M S215P Y224K E239G | 1.82 |
| G197S S215P V222I Y224K F230Y T231V W232Y R236K E239G | 1.82 |
| M336L I378K | 1.81 |
| G197D W232Y R236K E239G | 1.81 |
| L178K Y224K R236K | 1.80 |
| L178K G197E | 1.79 |
| L178K V222I Y224K T231V W232Y R236K F237Y E239G | 1.79 |
| I378K A394G | 1.79 |
| D186T G197E S215P Y224K R236K E239G | 1.75 |
| L178K S215P Y224K R236K | 1.75 |
| L178K K182D V222I Y224K | 1.75 |
| T368Y A394G | 1.75 |
| L178K K182D D200K Y224K T231V E239G | 1.75 |
| L178K R236K F237Y E239G | 1.74 |
| K182D G197D S215P R236K E239G | 1.73 |
| Q180M S215P V222I Y224K T231V W232Y R236K E239G | 1.72 |
| V222I Y224K R236K | 1.72 |
| G197E R236K | 1.71 |
| L178K V222I F230Y R236K E239G | 1.71 |
| D200K Y224K | 1.70 |
| G197D R236K | 1.69 |
| L178K G197S E239G | 1.69 |
| G197D Y224K W232Y E239G | 1.68 |
| S215P V222I Y224K T231V W232Y R236K F237Y E239G | 1.68 |
| K182D G197E T231V R236K F237Y E239G | 1.68 |
| Q180M D200K T231V R236K E239G | 1.68 |
| I348V A394G | 1.67 |
| L178K K182D D200K S215P R236K | 1.67 |
| D200K V222I R236K E239G | 1.66 |
| L178K D200K Y224K T231V E239G | 1.66 |
| Q180M S215P Y224K T231V W232Y R236K E239G | 1.65 |
| L178K V222I R236K | 1.65 |
| L178K S215P V222I Y224K F230Y T231V W232Y R236K F237Y | 1.65 |
| E239G | |
| Q180M Y224K E239G | 1.64 |
| V222I Y224K T231V R236K | 1.63 |
| G197B Y224K T231V | 1.62 |
| L178K K182D S215P V222I Y224K F230Y T231V W232Y R236K | 1.61 |
| E239G | |
| G197D T231V | 1.60 |
| Q171S I172V L178K I179L Q180M K182D Y224K T231V | 1.59 |
| V222I Y224K R236K E239G | 1.58 |
| Q180M R236K E239G | 1.57 |
| G191A G197S S215P W232Y E239G | 1.57 |
| I172V D200K Y224K | 1.56 |
| T368M Y369R | 1.56 |
| L178K Y224K T231V R236K | 1.55 |
| L178K Y224K R236K E239G | 1.55 |
| L178K G191A G197E V222I W232Y F237Y E239G | 1.54 |
| G197E S215P R236K | 1.54 |
| L178K K182D Y224K R236K E239G | 1.54 |
| L178K D200K E239G | 1.53 |
| L178K K182D Y224K E239G | 1.53 |
| D186T G197S | 1.53 |
| G197E S215P R236K E239G | 1.53 |
| G197E W232Y E239G | 1.52 |
| G197S Y224K T231V W232Y R236K F237Y E239G | 1.51 |
| G197S Y224K | 1.50 |
| D200K S215P V222I Y224K T231V W232Y R236K E239G | 1.50 |
| L178K K182D S215P Y224K R236K E239G | 1.50 |
| Q180M V222I Y224K T231V W232Y R236K E239G | 1.50 |
| L178K K182D S215P V222I R236K E239G | 1.50 |
| V222I Y224K F230Y T231V W232Y R236K F237Y E239G | 1.50 |
| D186T Y224K E239G | 1.49 |
| Q180M G197D W232Y E239G | 1.49 |
| L178K I179L Q180M F181L K182D | 1.49 |
| D200K V222I Y224K T231V | 1.48 |
| D186T T231V R236K E239G | 1.48 |
| G197S S215P E239G | 1.48 |
| S215P V222I Y224K R236K | 1.48 |
| G197E T231V | 1.48 |
| L178K I179L Q180M F181L K182D Y224K T231V E239G | 1.48 |
| L178K K182D S215P R236K E239G | 1.48 |
| L178K Q180M S215P V222I | 1.47 |
| V222I Y224K T231V W232Y E239G | 1.47 |
| Q180M Y224K T231V E239G | 1.47 |
| Q180M G197D | 1.47 |
| L178K K182D Y224K T231V | 1.46 |
| G191A Y224K T231V R236K E239G | 1.46 |
| D200K Y224K T231V E239G | 1.45 |
| L178K Y224K F230Y T231V W232Y R236K F237Y E239G | 1.45 |
| T373H A394G | 1.45 |
| L178K K182D S215P V222I T231V W232Y R236K E239G | 1.45 |
| L178K K182D V222I R236K E239G | 1.44 |
| D341I A394G | 1.44 |
| K182D G197E V222I E239G | 1.44 |
| D186T S215P E239G | 1.43 |
| L178K G197E E239G | 1.43 |
| L178K Y224K E239G | 1.43 |
| D200K V222I Y224K W232Y R236K E239G | 1.42 |
| I172V I173V G197D | 1.42 |
| D200K Y224K T231V W232Y R236K E239G | 1.42 |
| L178K Y224K W232Y R236K E239G | 1.41 |
| I172V D186T G197E W232Y E239G | 1.40 |
| S215P V222I R236K | 1.39 |
| Q180M Y224K | 1.39 |
| L178K S215P V222I R236K E239G | 1.39 |
| G197E S215P V222I | 1.39 |
| T368F T373H | 1.38 |
| G197E S215P E239G | 1.38 |
| Q171L I172V I173V L178K I179L Q180M K182D G197S | 1.38 |
| L178K Q180M S215P Y224K E239G | 1.37 |
| Q171S I172V L178K I179L Q180M K182D G197E F230Y | 1.36 |
| V222I Y224K T231V W232Y R236K F237Y E239G | 1.36 |
| G197E S21SP Y224K | 1.35 |
| D200K Y224K E239G | 1.35 |
| L178K K182D V222I Y224K F230Y T231V W232Y R236K E239G | 1.35 |
| L178K K182D R236K E239G | 1.35 |
| L178K K182D D200K S215P Y224K T231V W232Y E239G | 1.34 |
| I172V L178K S21SP V222I Y224K | 1.34 |
| G191A S215P V222I E239G | 1.34 |
| G197S V222I T231V R236K E239G | 1.33 |
| Q171S I179L G197D Y224K | 1.33 |
| I172V V222I Y224K F230Y T231V W232Y R236K E239G | 1.33 |
| L178K K182D G191A E239G | 1.32 |
| D186T S21SP T231V R236K | 1.32 |
| L178K G191A S21SP T231V R236K E239G | 1.31 |
| L178K Y224K T231V W232Y R236K E239G | 1.30 |
| G197E V222I T231V | 1.30 |
| D186T Y224K T231V W232Y R236K E239G | 1.29 |
| S215P V222I R236K E239G | 1.29 |
| L178K K182D D200K T231V R236K E239G | 1.29 |
| L178K K182D D200K S215P R236K E239G | 1.29 |
| L178K V222I R236K E239G | 1.29 |
| G191A G197S W232Y E239G | 1.29 |
| L178K K182D G191A G197S S215P F230Y | 1.29 |
| G197E E239G | 1.29 |
| D341I T368F | 1.28 |
| T372E I378K | 1.28 |
| V222I Y224K W232Y R236K E239G | 1.28 |
| L178K S215P Y224K T231V W232Y R236K F237Y E239G | 1.27 |
| V222I T231V R236K E239G | 1.27 |
| L178K S21SP V222I Y224K T231V W232Y R236K E239G | 1.27 |
| S215P Y224K T231V R236K | 1.27 |
| G191A G197E R236K | 1.27 |
| L178K K182D Y224K | 1.27 |
| L178K K182D G191A R236K E239G | 1.26 |
| S215P V222I Y224K | 1.26 |
| G197E T231V E239G | 1.26 |
| V222I Y224K | 1.26 |
| V222I R236K F237Y E239G | 1.25 |
| Q171S I172V L178K I179L Q180M K182D | 1.25 |
| I172V G197E S215P E239G | 1.24 |
| L178K K182D Y224K T231V W232Y R236K E239G | 1.24 |
| L178K K182D V222I R236K | 1.24 |
| L178K K182D R236K F237Y E239G | 1.24 |
| G197E F230Y T231V W232Y R236K F237Y E239G | 1.23 |
| L178K S215P R236K F237Y E239G | 1.23 |
| D200K S215P V222I Y224K F230Y T231V W232Y E239G | 1.23 |
| L178K K182D Y224K W232Y E239G | 1.23 |
| I172V V222I Y224K F230Y T231V W232Y R236K F237Y E239G | 1.23 |
| Y224K R236K | 1.22 |
| L178K V222I Y224K W232Y E239G | 1.22 |
| V222I R236K | 1.21 |
| I172V Q180M W232Y E239G | 1.21 |
| Q171L G197D S215P | 1.20 |
| G197E S215P | 1.20 |
| S215P Y224K R236K E239G | 1.20 |
| G197E V222I W232Y | 1.20 |
| I172V L178K G197S V222I Y224K T231V W232Y E239G | 1.20 |
| Y369R A394G | 1.19 |
| L178K K182D V222I E239G | 1.19 |
| L178K V222I E239G | 1.19 |
| D200K V222I E239G | 1.19 |
| S215P V222I Y224K F230Y T231V W232Y R236K F237Y E239G | 1.19 |
| L178K S215P V222I Y224K T231V W232Y E239G | 1.18 |
| L178K K182D S215P Y224K | 1.18 |
| L178K G197S T231V W232Y E239G | 1.18 |
| I172V S215P V222I Y224K F230Y T231V W232Y R236K F237Y | 1.17 |
| E239G | |
| Q171L I172V I173V L178K I179L Q180M K182D R236K E239G | 1.17 |
| Y224K T231V R236K E239G | 1.17 |
| L178K Y224K F230Y T231V W232Y R236K E239G | 1.16 |
| D200K Y224K W232Y E239G | 1.16 |
| G197D W232Y E239G | 1.16 |
| L178K K182D S215P R236K | 1.15 |
| D186T E239G | 1.15 |
| L178K K182D S215P W232Y R236K E239G | 1.15 |
| K182D G197D | 1.14 |
| Y224K R236K E239G | 1.14 |
| D200K S215P Y224K | 1.13 |
| D200K S215P T231V R236K E239G | 1.13 |
| G197E W232Y | 1.13 |
| G197S T231V W232Y E239G | 1.13 |
| L178K S215P Y224K T231V W232Y R236K E239G | 1.12 |
| L178K K182D S215P Y224K R236K | 1.12 |
| G191A G197E S215P V222I E239G | 1.12 |
| L178K I179L Q180M F181L K182D E239G | 1.12 |
| L178K D200K Y224K W232Y E239G | 1.12 |
| L178K K182D T231V E239G | 1.12 |
| V222I R236K E239G | 1.11 |
| S215P V222I Y224K R236K E239G | 1.11 |
| S215P V222I Y224K T231V W232Y R236K E239G | 1.11 |
| V222I Y224K T231V R236K E239G | 1.11 |
| L178K K182D S215P V222I W232Y E239G | 1.10 |
| Q180M E239G | 1.09 |
| G191A Y224K R236K E239G | 1.09 |
| G191A G197E S215P V222I Y224K F230Y T231V W232Y R236K | 1.09 |
| E239G | |
| L178K S215P V222I Y224K E239G | 1.09 |
| Q180M S215P Y224K F230Y T231V W232Y R236K E239G | 1.08 |
| S215P Y224K T231V R236K F237Y E239G | 1.08 |
| L178K V222I Y224K | 1.08 |
| I179L Q180M E239G | 1.07 |
| Q171S G197S | 1.07 |
| G197E F230Y T231V | 1.07 |
| L178K S215P V222I T231V | 1.07 |
| S215P V222I Y224K W232Y E239G | 1.07 |
| K182D G197S S21SP Y224K T231V W232Y R236K E239G | 1.07 |
| Q171S G197D | 1.06 |
| D200K S215P R236K E239G | 1.06 |
| V222I Y224K F230Y T231V W232Y R236K E239G | 1.06 |
| K182D G197E E239G | 1.06 |
| L178K Y224K T231V | 1.05 |
| L178K S21SP Y224K R236K E239G | 1.05 |
| Q171L I172V L178K I179L Q180M K182D Y224K W232Y E239G | 1.05 |
| G197D Y224K W232Y | 1.05 |
| D200K S215P Y224K E239G | 1.04 |
| G197D W232Y | 1.04 |
| Q171S S215P V222I Y224K T231V W232Y R236K F237Y E239G | 1.03 |
| L178K Y224K T231V W232Y E239G | 1.02 |
| T373H I378K | 1.02 |
| D200K S215P V222I Y224K F230Y T231V W232Y R236K E239G | 1.02 |
| V222I Y224K E239G | 1.02 |
| K182D G191A G197E | 1.01 |
| G197D V222I W232Y | 1.01 |
| L178K Y224K | 1.01 |

### Example 2 Arrayed Screening of Phi29 Candidates for Increased dsDNA at Elevated Temperatures

### Methods

### Cloning and Overnight Culture

Candidate Phi29 variants identified from the selection process and subsequence sequencing were synthesized, cloned into expression plasmids and transformed into competent Escherichia coli cells. Individual colonies were picked and inoculated into 1-2 mL deep-well plates, each well containing 300 μL LB medium supplemented with carbenicillin (100 igg/mL). Plates were sealed with breathable membranes and incubated overnight at 37° C. with shaking at 1500 rpm.

### Pre-Growth, Glycerol Stock Preparation, and Induction

Following overnight incubation, a 100-fold dilution was performed by transferring 3 μL of each overnight culture into fresh wells containing 297 μL LB+Crb medium. Concurrently, glycerol stocks were prepared by adding 297 μL of 40% glycerol to remaining overnight cultures, sealing with −80° C. foil film, and storing at −80° C. for future use. The diluted cultures were incubated for 3 hours at 37° C., shaking at 15(0) rpm, followed by induction with IPTG at a final concentration of 1 mM. Plates were resealed and incubated for an additional 3 hours at 30° C.

### Arrayed Lysis of Induced Cells

After induction, aliquots of each induced culture were transferred into new 1 mL deep-well plates. Plates were centrifuged at maximum speed for 10 minutes. Supernatants were aspirated and discarded, and plates were sealed and stored briefly at −80° C. Plates were then thawed at room temperature, followed by the addition of 100 μL NEBExpress E. coli Lysis Reagent supplemented with lysozyme at 10 μg/mL. Lysis was performed on an unheated benchtop plate shaker at 1500 rpm for 20 minutes at room temperature. Lysates were immediately placed on ice following lysis.

### Multiple Displacement Amplification (MDA) Reaction Setup

MDA reactions were set up to assess polymerase activity at elevated temperatures. Master mixes for reactions were prepared with the following final concentrations per reaction:
1. 1× Phi29 reaction buffer
2. 1 mM each dNTP
3. 50 μM exonuclease-resistant random primer mix
4. 2× Qbit dye concentration for enhanced sensitivity
5. 10 ng pUC19 plasmid DNA as supplementary template

For each reaction, 2 μL of freshly prepared cell lysate was added to the master mix to achieve a total reaction volume of 20 μL. Plates were gently mixed by pipetting and aliquoted into qPCR-compatible plates. Plates were sealed with qPCR films and subjected to incubation and real-time monitoring at the desired elevated temperatures to quantify and identify Phi29 DNA polymerase variants with improved dsDNA synthesis capabilities.

Polymerase variants showing superior performance in synthesizing double-stranded DNA at elevated temperatures were selected for further characterization and validation.

### Results

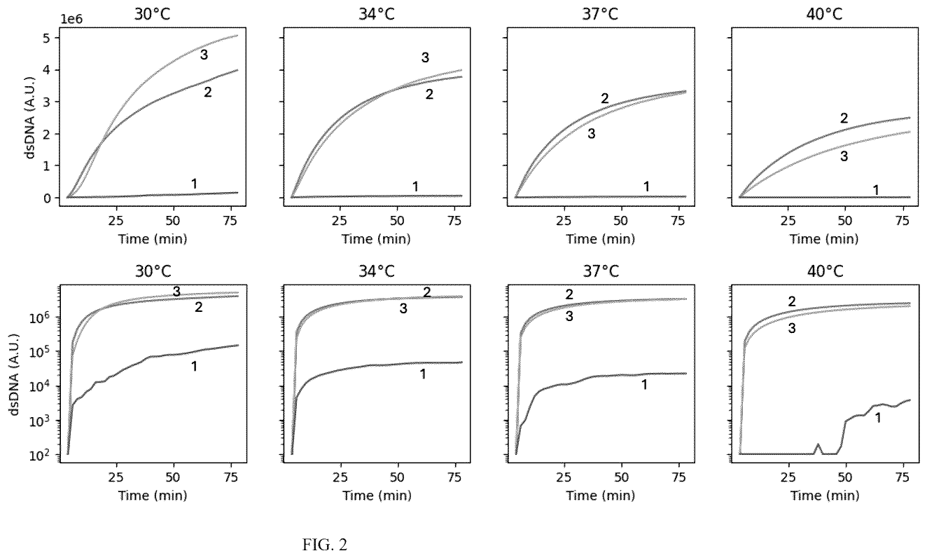

As shown in FIG. 2, DNA polymerases having the amino acid sequences of SEQ ID NO: 2 and SEQ ID NO: 3 showed much higher yield in DNA amplification and higher thermostability compared with wild type Phi29 DNA polymerase (having the amino acid sequence of SEQ ID NO: 1) under 30° C., 34° C., 37° C. and 40° C. in the time course show-n. This was demonstrated using e coli cell lysate-based expression.

### Example 3 Mutants Outperform WT Phi29 in RCA Experiments

### Methods

Using cell-free expression kit PUREfrex 2.0, proteins were expressed. The activity of enzymes was tested using a standard RCA assay, in which 10 ng of puc19 plasmid was used as template. The RCA reactions were incubated at 37° C. and 42° C. and measurements were taken every 2 mins.

### Results

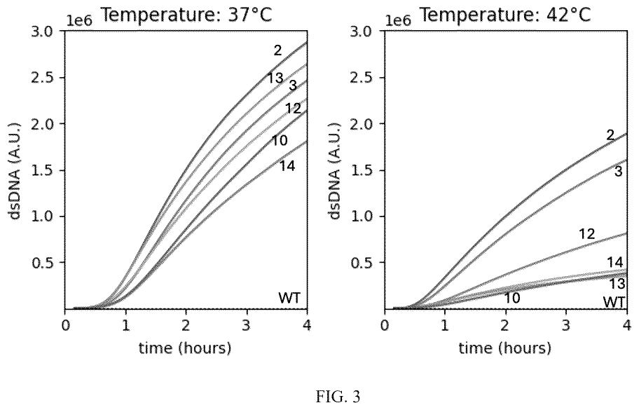

We observed multiple sequences that outperform wild type Phi29 DNA polymerase (SEQ ID NO. 1) in DNA amplification experiments under both 37° C. and 42° C. As shown in FIG. 3, under both 37° C. and 42° C., SEQ ID NO. 1 did not show visible activity (shown as the baseline in the figure), as its working temperature is 30° C. and becomes unstable under 37° C. and 42° C. DNA polymerases having the amino acid sequences of SEQ ID NO: 2, 3, 10, 12, 13, and 14 all produced higher levels of dsDNA synthesis than did wild-type Phi29 DNA polymerase at 37° C. and 42° C. at least as soon as within 1 hour, at which time dsDNA produced by wilt type Phi29 DNA polymerase was essentially undetectable.

Thus, a DNA polymerase as disclosed herein exhibits higher yield than wild type Phi29. For example, at about 30° C., at about 75 mins MDA reaction. DNA polymerases having amino acids with sequences as set out in SEQ ID NO: 2 and SEQ ID NO: 3 made about 10× more total amount of dsDNA than wild type Phi29 under the same condition (FIG. 3, Lower left 1). A DNA polymerase may have higher yield than wild type Phi29 having the amino acid sequence as set out in SEQ ID NO: I when, at about 30° C., at about 75 mins MDA reaction, it makes at least about 1× more total amount of dsDNA than wild type Phi29 under the same conditions.

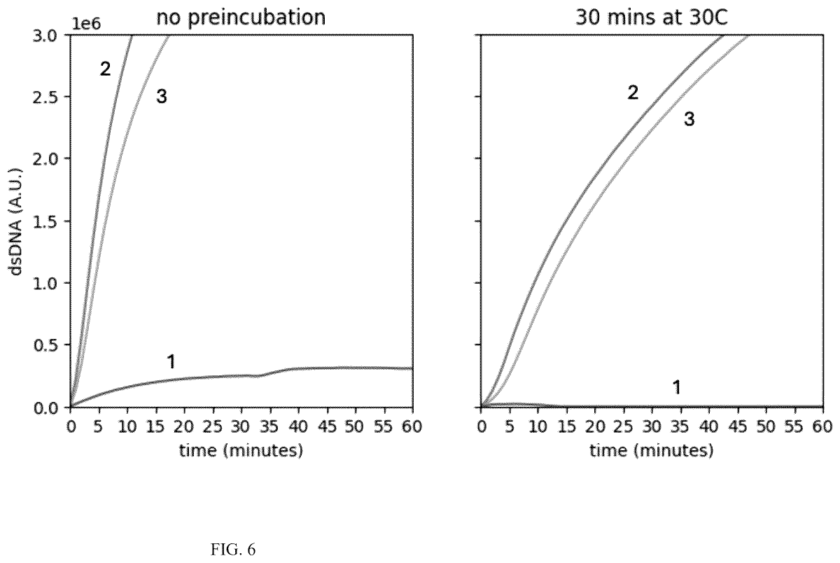

As the temperature increase to 34° C., 37° C. and 40° C., DNA polymerases having the amino acid sequence as set out in SEQ ID NO: 2 and SEQ ID NO: 3 still made similar amount of dsDNA (>10⁶ A.U.) as they did at °30C. However the Phi29 DNA polymerase having the amino acid sequence as set out in SEQ ID NO: I had reduced performance from 10⁵ A.U. under 30° C. to 104 under 37° C. and was inactive at 40° C. (FIG. 3, Lower panels). Thus, a DNA polymerase as disclosed herein may have higher thermostability than a Phi29 DNA polymerase having the amino acid sequence set out in SEQ ID NO: 1 when, in an implementation of having higher thermostability, the amount of dsDNA synthesized by the DNA polymerase in an MDA reaction at 37° C. is not at least 10% less than the amount of dsDNA synthesized by the DNA polymerase in an MDA reaction at 37° C., whereas the amount of dsDNA synthesized by a Phi29 DNA polymerase having the amino acid sequence as set out in SEQ ID NO: 1 in an MDA reaction at 37° C. is at least 50% less than the amount of dsDNA synthesized by the Phi29 DNA polymerase in an MDA reaction at 37° C., other conditions being the same. Another way in which a DNA polymerase as described herein may have higher thermostability than a DNA polymerase having the amino acid sequence as set out in SEQ ID NO: 1 (wild type Phi29) is described in relation to examples depicted in FIG. 6 herein. A DNA polymerase as described herein has higher thermostability than a DNA polymerase having the amino acid sequence as set out in SEQ ID NO: 1 (wild type Phi29) if it satisfies the description of higher thermostability as given in the present paragraph or if it satisfies the description of higher thermostability as given in relation to the examples depicted in FIG. 6 herein.

### Example 4 Mutants Outperform WT Phi29 in DNA Amplification (RCA) Experiments

### Methods

Phi29 DNA Polymerase and various mutants were expressed using the cell-free expression kit PureFREX 2.0 and used to perform RCA at 37C and 40C via the following protocol:

TABLE 7
Prepare the following amplification reaction mixture:

| | For each reaction |
|---|---|
| 10x Phi29 reaction buffer | 2 uL |
| 10 mM (each base) dNTP mix | 2 uL |
| 500 uM Exo-resistant Random | 2 uL |
| Primers | |
| 10 ng/uL Puc19 Plasmid DNA | 1 uL |
| 40x Qubit Fluorescent | 1 uL |
| dsDNA Dye | |
| CFPS-produced Phi29 DNA | 2 uL |
| Polymerase | |
| Water | 10 uL |

(10× Phi29 reaction buffer: 330 mM Tris-acetate, pH 7.9; 100 mM magnesium acetate, 660 mM potassium acetate, 1% Tween 20, 10 mM DTT)

Incubate the RCA reactions at 37° C. and 40° C. for 4 hours in a qPCR machine measuring Qubit dsDNA fluorescence. To gauge RCA reaction efficiency, qPCR curves detecting Qubit Fluorescent dsDNA dye were analyzed.

### Results

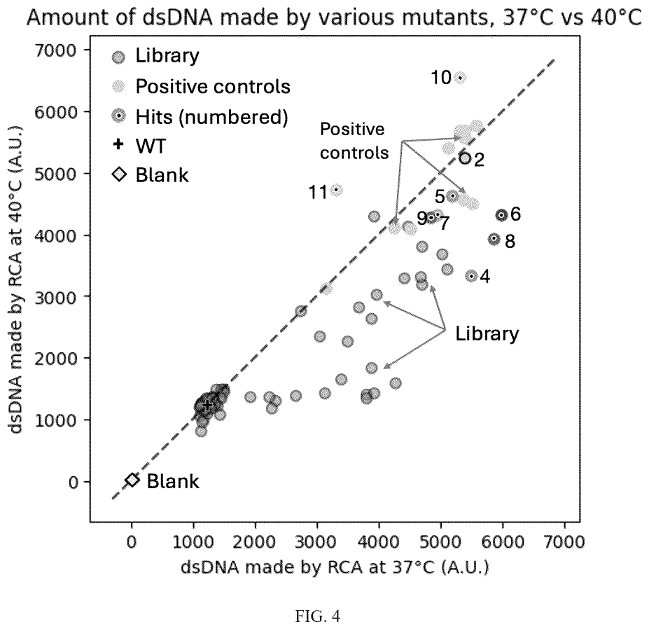

Nine sequences tested in the library showed high dsDNA yield comparable to the positive controls, while WT Phi29 and many mutants tested in the library had no DNA yield under 37° C. and 40° C. conditions. Most of the sequences tested in the library showed higher dsDNA yield under 37° C. than under 40° C. In general, we observed a dsDNA yield correlation between the 37° C. and 40° C. experiments. See FIG. 4.

### Example 5 (e547) Mutants Outperform WT Phi29 in Protein Solubility and Manufacturability in E. coli

### Methods

Enzymes were expressed and induced in strain B121 DE3. Cells were lysed using NEB lysis buffer and lysozyme. Uninduced and induced cells were lysed and spun down. Aspirated supernatant were loaded to SDS page.

### Results

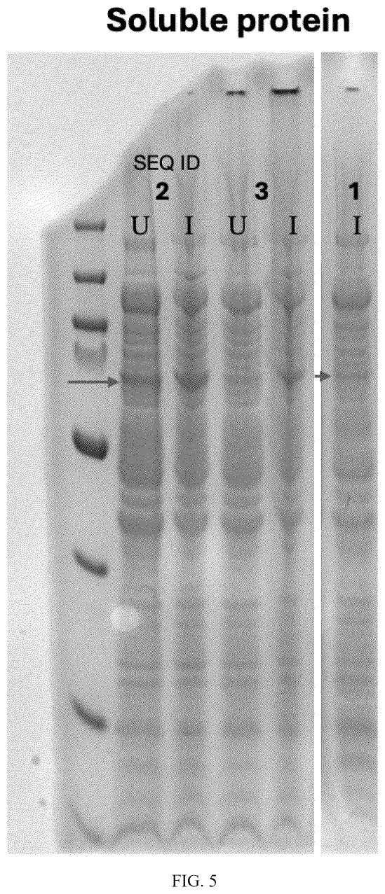

As shown in FIG. 5, DNA polymerases having the amino acid sequences of SEQ ID NOs: 2 and 3 have higher soluble expression in E. coli compared with wild type Phi29 DNA polymerase (SEQ ID NO: 1). After induction, DNA polymerases having the amino acid sequences as set out in SEQ ID NO: 2 and SEQ ID NO: 3 had higher solubility than wild type Phi29 DNA polymerase, in that they yielded at least about twice as much (>about 100% more) soluble protein expression in supernatant after spinning down than wild type, with wild type expression significantly lower.

### Example 6 Mutants Outperform WT Phi29 in Thermostability

### Methods

Enzymes were incubated in lysate, buffer & template (10 ng pUC19) for 0 or 30 minutes at 30° C., then dNTPs, random primers and Qubit dye were added, and the reaction moved to the qPCR machine for an RCA reaction at 30° C.

Improved Phi29 thermostability was evaluated by measuring residual amplification activity after preincubation in the presence of substrate at a range of temperatures. Enzymes with more residual activity after incubation at higher temperatures and/or longer periods of time indicate higher stability.

Two mutant variants of Phi29 DNA Polymerase were constructed:

In this case, the half-life of different Phi29 DNA polymerase mutants in the presence of substrate was measured using the following protocol.

Cells expressing Phi29 and two Phi29 mutants were lysed with 100 uL lysis buffer supplemented with 10 ug/mL lysozyme. Lysates were then spun down at 15000rcf for 15 minutes at 4C. The supernatant was aspirated and kept on ice.

TABLE 8
Prepare the following reaction mixture:

| | For each reaction |
|---|---|
| 10x Phi29 reaction buffer | 1 uL |
| 10 ng/uL Puc19 Plasmid DNA | 1 uL |
| Crude Phi29 lysate | 2 uL |
| H2O | 6 uL |

(10× Phi29 reaction buffer: 330 mM Tris-acetate, pH 7.9; 100 mM magnesium acetate, 660 mM potassium acetate, 1% Tween 20, 10 mM DTT)

Incubate reactions at 30C for 0 minutes and 30 minutes

After incubation, place samples in an ice bath and add 10 uL Post-Incubation Solution

| Post-Incubation Solution | For each reaction |
|---|---|
| 10x Phi29 Reaction Buffer | 1 uL |
| 500 uM Exo-resistant Random | 2 uL |
| Primers | |
| 10 mM (each base) dNTP mix | 2 uL |
| 40x Qubit Fluorescent DNA | 1 uL |
| Dye | |
| H2O | 4 uL |

Incubate samples at 30C for 2 hours in a qPCR machine.

To gauge MDA reaction efficiency, qPCR curves detecting Qubit Fluorescent DNA dye were analyzed.

### Results

FIG. 6 shows multiple displacement amplification (MDA) reaction results for DNA polymerases having the amino acid sequences set out in SEQ ID NOs: 1 (wild-type Phi29 DNA polymerase), 2, and 3, with and without 30 min 30° C. preincubation. Activity of enzymes having the amino acid sequences of SEQ ID NO: 2 and SEQ ID NO: 3 with or without preincubation showed significantly higher activity than wild type Phi29 (SEQ ID NO: 1). After 30 min preincubation at 30° C., a DNA polymerase having the amino acid sequence of SEQ ID NO: 1 (wild-type Phi29) lost its activity entirely, whereas DNA polymerases having the amino acid sequences as set out in SEQ ID NO: 2 and SEQ ID NO: 3 maintained the majority of their activities and produced 10⁶ A.U. dsDNA in 10 nuns.

Thus, a DNA polymerase as disclosed herein may have higher thermostability than a Phi29 DNA polymerase having the amino acid sequence set out in SEQ ID NO: I when, in an implementation of having higher thermostability, the amount of dsDNA synthesized by a DNA polymerase after about 15 min of its use in an MDA reaction at about 37° C. following preincubation of the DNA polymerase at about 30° C. for about 30 min before the MDA reaction, is at least about 30% of the amount of dsDNA synthesized by a DNA polymerase having the same amino acid sequence after about 10 min of its use in an MDA reaction at about 37° C., without preincubation of the DNA polymerase at about 30° C. before the MDA reaction, whereas the amount of dsDNA synthesized by a DNA polymerase having the amino acid sequence set out in SEQ ID NO: 1 (wild type Phi29) after about 15 min of its use in an MDA reaction at about 37° C. following preincubation of the DNA polymerase at about 30° C. for about 30 min before the MDA reaction, is less by at least about 95% that the amount of dsDNA synthesized thereby after about 15 min of its use in an MDA reaction at about 37° C. without preincubation thereof at about 30° C. before the MDA reaction, with the conditions of the preincubation and MDA of the DNA polymerases otherwise being consistent with each other.

Another way in which a DNA polymerase as described herein may have higher thermostability than a DNA polymerase having the amino acid sequence as set out in SEQ ID NO: 1 (wild type Phi29) is described in relation to examples depicted in FIG. 3 herein. A DNA polymerase as described herein has higher thermostability than a DNA polymerase having the amino acid sequence as set out in SEQ ID NO: 1 (wild type Phi29) if it satisfies the description of higher thermostability as given in the previous paragraph or if it satisfies the description of higher thermostability as given in relation to the examples depicted in FIG. 3 herein.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein and may be used to achieve the benefits and advantages described herein.

Sequences
SEQ ID NO: 1
MKHMPRKMYS CDFETTTKVE DCRVWAYGYM NIEDHSEYKI GNSLDEFMAW 50
VLKVQADLYF HNLKEDGAFI INWLERNGFK WSADGLPNTY NTIISRMGQW 100
YMIDICLGYK GKRKIHTVIY DSLKKLPFPV KKIAKDFKLT VLKGDIDYHK 150
ERPVGYKITP EEYAYIKNDI QIIAEALLIQ FKQGLDRMTA GSDSLKGFKD 200
IITTKKFKKV FPTLSLGLDK EVRYAYRGGF TWINDRFKEK EIGEGMVFDV 250
NSLYPAQMYS RLLPYGEPIV FEGKYVWDED YPLHIQHIRC EFELKEGYIP 300
TIQIKRSREY KGNEYLKSSG GEIADLWLSN VDLELMKEHY DLYNVEYISG 350
LKFKATTGLE KDFIDKWTYI KTTSEGAIKQ LAKLMLNSLY GKFASNPDVT 400
GKVPYLKENG ALGFRIGEEE TKDPVYTPMG VFITAWARYT TITAAQACYD 450
RIIYCDTDSI HITGTEIPDV IKDIVDPKKL GYWAHESTEK RAKYLRQKTY 500
IQDIYMKEVD GKLVEGSPDD YTDIKFSVKC AGMTDKIKKE VTFENEKVGF 550
SRKMKPKPVQ VPGGVVLVDD TFTIK
SEQ ID NO: 2
MKHMPRKMYS CDFETTTKVE DCRVWAYGYM NIEDHSEYKI GNSLDEFMAW 50
VLKVQADLYF HNLKEDGAFI INWLERNGFK WSADGLPNTY NTIISRMGQW 100
YMIDICIGYK GKRKIHTVIY DSLKKLPFPV KKIAKDFKLT VLKGDIDYHK 150
ERPVGYKITP EEYAYIKNDI QIIAEALKLM FDQGLDRMTA GSDSLKGFKD 200
IITTKKFKKV FPTLSLGLDK EVRKAYRGGF TWLNDKFKEK EIGEGMVEDV 250
NSLYPAQMYS RLLPYGEPIV FEGKYVWDED YPLHIQHIRC EFELKEGYIP 300
TIQIKRSRFY KGNEYLKSSG GEIADLWLSN VDLELLKEHY KLYNVEYVRG 350
LKFKATTGLF KDFIDKWYYI KTHSEGAKKQ LAKLMLNSLY GKFGSNPDVT 400
GKVPYLKENG ALGERIGEEE TKDPVYTPMG VFITAWARYT TITAAQACYD 450
RIIYCDTDSI HLTGTEIPDV IKDIVDPKKL GYWAHESTEK RAKYLRQKTY 500
IQDIYMKEVD GKLVEGSPDD YTDIKFSVKC AGMTDKIKKE VTFENFKVGF 550
SRKMKPKPVQ VPGGVVLVDD TFTIK
SEQ ID NO: 3
MKHMPRKMYS CDFETTTKVE DCRVWAYGYM NIEDHSEYKI GNSLDEFMAW 50
VLKVQADLYF HNLKFDGAFI INWLERNGFK WSADGLPNTY NTIISRMGQW 100
YMIDICIGYK GKHKIHTVIY DSLKKLPFPV KKIAKDFKLT VLKGDIDYHK 150
ERPVGYKITP EEYAYIKNDI QIIAEALKLM FDQGLDRMTA GSDSLKGFKD 200
IITTKKFKKV FPTLSLGLDK EVRKAYRGGF TWLNDKFKEK EIGEGMVEDV 250
NSLYPAQMYS RLLPYGEPIV FEGKYVWDED YPLHIQHIRC EFELKEGYIP 300
TIQIKRSREY KGNEYLKSSG GEIADLWISN VDLELLKEHY KLYNVEYVRG 350
LKFKATTGLE KDFIDKWYYI KTHSEGAKKQ LAKLMLNSLY GKFGSNPDVT 400
GKVPYLKENG ALGFRLGEEE TKDPVYTPMG VFITAWARYT TITAAQACYD 450
RIIYCDTDSI HITGTEIPDV IKDIVDPKKL GYWAHESTEK RAKYLRQKTY 500
IQDIYMKEVD GKLVEGSPDD YTDIKFSVKC AGMTDKIKKE VTFENFKVGF 550
SRKMKPKPVQ VPGGVVLVDD TFTIK
SEQ ID NO: 4
MKHMPRKMYS CDFETTTKVE DCRVWAYGYM NIEDHSEYKI GNSIDEFMAW 50
VLKVQADLYF HNLKFDGAFI INWLERNGFK WSADGLPNTY NTIISRMGQW 100
YMIDICIGYK GKRKIHTLIY DSLKKLPFPV KKIAKDFKLT VLKGDIDYNK 150
ERPVGYKITP EEYAYIKNDI QIIAEALKIM FDQGLDRMTA GSDSLKGFKD 200
IITTKKFKKV FPTLSLGLDK EVRKAYRGGF TWLNDKEKEK EIGEGMVFDV 250
NSLYPAQMYS RLLPYGEPIV FEGKYVWDED YPLHIQHIRC EFELKEGYIP 300
TIQIKRSRFY KGNEYLKSSG GEIADLWLSN VDLELLKEHY KLYNVEYVRG 350
LKFKATTGLF KDFIDKWYYI KTHSEGAKKQ LAKLMLNSLY GKFGSNPDVT 400
GKVPYLKENG ALGERLGEEE TKDPVYTPMG VFITAWARYT TITAAQACYD 450
RIIYCDTDSI HITGTEIPDV IKDIVDPKKL GYWAHESTEK RAKYLRQKTY 500
IQDIYMKEVD GKLVEGSPDD YTDIKFSVKC AGMTDKIKKE VTFENFKVGF 550
SRKMKPKPVQ VPGGVVLVDD TFTIK
SEQ ID NO: 5
MKHMPRKMYS CDFETTTKVE DCRVWAYGYM NIEDHSEYKI GNSLDEFMAW 50
VLKVQADLYE HNLKEDGAFI INWLERNGFK WSADGLPNTY NTIISRMGQW 100
YMIDICIGYK GKRKIHTVIY DSLKKLPFPV KKIAKDFKLT VLKGNIDYLK 150
ERPVGYKITP EEYAYIKNDI QIIAEALKLM FDQGLDRMTA GSDSLKGFKD 200
IITTKKFKKV FPTLSLGLDK EVRKAYRGGF TWLNDKFKEK EIGEGMVEDV 250
NSLYPAQMYS RILPYGEPIV FEGKYVWDED YPLHIQHIRC EFELKEGYIP 300
TIQIKRSRFY KGNEYLKSSG GEIADLWLSN VDLELLKEHY KLYNVEYVRG 350
LKFKATTGLF KDFIDKWYYI KTHSEGAKKQ LAKLMLNSLY GKFGSNPDVT 400
GKVPYLKENG ALGFRIGEEE TKDPVYTPMG VFITAWARYT TITAAQACYD 450
RIIYCDTDSI HLTGTEIPDV IKDIVDPKKL GYWAHESTEK RAKYLRQKTY 500
IQDIYMKEVD GKLVEGSPDD YTDIKFSVKC AGMTDKIKKE VTFENFKVGF 550
SRKMKPKPVQ VPGGVVLVDD TETIK
SEQ ID NO: 6
MKHMPRKMYS CDFETTTKVE DCRVWAYGYM NIEDHSEYKI GNSLDEFMAW 50
VLKVQADLYF HNLKEDGAFI INWLERNGFK WSADGLPNTY NTIISRMGQW 100
YMIDICIGYK GKHKIHTSIY DSLKKLPFPV KKIAKDFKLT VLKGNIDYHK 150
ERPVGYKITP EEYAYIKNDI QIVAEALKLM FDQGLDRMTA GSDSLKGFKD 200
IITTKKFKKV FPTLSLGLDK EVRKAYRGGF TWLNDKFKEK EIGEGMVEDV 250
NSLYPAQMYS RLLPYGEPIV FEGKYVWDED YPLHIQHIRC EFEIKEGYIP 300
TIQIKRSRFY KGNEYLKSSG GEIADLWISN VDLELLKEHY KLYNVEYVRG 350
LKFKATTGLF KDFIDKWYYI KTHSEGAKKQ LAKLMLNSLY GKFGSNPDVT 400
GKVPYLKENG ALGERLGEEE TKDPVYTPMG VFITAWARYT TITAAQACYD 450
RIIYCDTDSI HITGTEIPDV IKDIVDPKKL GYWAHESTEK RAKYLRQKTY 500
IQDIYMKEVD GKLVEGSPDD YTDIKFSVKC AGMTDKIKKE VTFENFKVGF 550
SRKMKPKPVQ VPGGVVLVDD TFTIK
SEQ ID NO: 7
MKHMPRKMYS CDFETTIKVE DCRVWAYGYM NIEDHSEYKI GNSLDEFMAW 50
VLKVQADLYE HNLKEDGAFI INWLERNGEK WSADGLPNTY NTIISRMGQW 100
YMIDICIGYK GKRKIHTVIY DSLKKLPFPV KKIAKDFKLT VLKGDIDYHK 150
ERPVGYKITP EEYAYIKNDI LIIAEALKLM FDQGLDRMTA GSDSLKGFKD 200
IITTKKFKKV FPTLSLGLDK EVRKAYRGGF TWINDKFKEK EIGEGMVEDV 250
NSLYPAQMYS RLLPYGEPIV FEGKYVWDED YPLHIQHIRC EFELKEGYIP 300
TIQIKRSRFY KGNEYLKSSG GEIADLWLSN VDLELLKEHY KLYNVEYVRG 350
LKFKATTGLF KDFIDKWYYI KTHSEGAKKQ LAKLMLNSLY GKFGSNPDVT 400
GKVPYLKENG ALGFRIGEEE TKDPVYTPMG VFITAWARYT TITAAQACYD 450
RIIYCDTDSI HLTGTEIPDV IKDIVDPKKL GYWAHESTEK RAKYLRQKTY 500
IQDIYMKEVD GKLVEGSPDD YTDIKFSVKC AGMTDKIKKE VTFENFKVGF 550
SRKMKPKPVQ VPGGVVLVDD TETIK
SEQ ID NO: 8
MKHMPRKMYS CDFETTTKVE DCRVWAYGYM NIEDHSEYKI GNSLDEFMAW 50
VLKVQADLYF HNLKFDGAFI INWLERNGFK WSADGLPNTY NTIISRMGQW 100
YMIDICIGYK GKRKIHTSIY DSLKKLPFPV KKIAKDFKLT VLKGDIDYHK 150
ERPVGYKITP EEYAYIKNDI LIIAEALKIM FDQGLTRMTA GSDSLKGFKD 200
IITTKKFKKV FPTLSLGLDK EVRKAYRGGF TWLNDKFKEK EIGEGMVFDV 250
NSLYPAQMYS RLLPYGEPIV FEGKYVWDED YPLHIQHIRC EFELKEGYIP 300
TIQIKRSRFY KGNEYLKSSG GEIADLWLSN VDLELLKEHY KLYNVEYVRG 350
LKFKATTGLF KDFIDKWYYI KTHSEGAKKQ LAKLMLNSLY GKFGSNPDVT 400
GKVPYLKENG ALGERLGEEE TKDPVYTPMG VFITAWARYT TITAAQACYD 450
RIIYCDTDSI HLTGTEIPDV IKDIVDPKKL GYWAHESTEK RAKYLRQKTY 500
IQDIYMKEVD GKLVEGSPDD YTDIKFSVKC AGMTDKIKKE VTFENFKVGF 550
SRKMKPKPVQ VPGGVVLVDD TFTIK
SEQ ID NO: 9
MKHMPRKMYS CDFETTTKVE DCRVWAYGYM NIEDHSEYKI GNSLDEFMAW 50
VLKVQADLYF HNLKFDGAFI INWLERNGFK WSADGLPNTY NTIISRMGQW 100
YMIDICIGYK GKRKIHTVIY DSLKKLPFPV KKIAKDEKLT VLKGDIDYHK 150
ERPVGYKITP EEYAYIKNDI QIVAEALKLM FDQGLDRMTA GSDSLKGFKD 200
IITTKKFKKV FPTLSLGLDK EVRKAYRGGF TWLNDKFKEK EIGEGMVFDV 250
NSLYPAQMYS RLLPYGEPIV FEGKYVWDED YPLHIQHIRC EFELKEGYIP 300
TIQIKRSREY KGNEYLKSSG GEIADLWLSN VDLELLKEHY KLYNVEYVRG 350
LKFKATTGLE KDFIDKWYYI KTHSEGAKKQ LAKLMLNSLY GKFGSNPDVT 400
GKVPYLKENG ALGFRLGEEE TKDPVYTPMG VFITAWARYT TITAAQACYD 450
RIIYCDTDSI HLTGTEIPDV IKDIVDPKKL GYWAHESTEK RAKYLRQKTY 500
IQDIYMKEVD GKIVEGSPDD YTDIKFSVKC AGMTDKIKKE VTFENFKVGF 550
SRKMKPKPVQ VPGGVVLVDD TFTIK
SEQ ID NO: 10
MKHMPRKMYS CDFETTIKVE DCRVWAYGYM NIEDHSEYKI GNSIDEFMAW 50
VLKVQADLYF HNLKEDGAFI INWLERNGFK WSADGLPNTY NTIISRMGQW 100
YMIDICIGYK GKRKIHTVIY DSLKKLPFPV KKIAKDFKLT VLKGDIDYLK 150
ERPVGYKITP EEYAYIKNDI QIIAEALKLM FDQGLTRMTA GSDSLKGFKD 200
IITTKKFKKV FPTLSLGLDK EVRKAYRGGF TWINDKFKEK EIGEGMVEDV 250
NSLYPAQMYS RLLPYGEPIV FEGKYVWDED YPLHIQHIRC EFELKEGYIP 300
TIQIKRSRFY KGNEYLKSSG GEIADLWLSN VDLELLKEHY KLYNVEYVRG 350
LKFKATTGLE KDFIDKWYYI KTHSEGAKKQ LAKLMLNSLY GKFGSNPDVT 400
GKVPYLKENG ALGFRLGEEE TKDPVYT PMG VFITAWARYT TITAAQACYD 450
RIIYCDTDSI HLTGTEIPDV IKDIVDPKKL GYWAHESTEK RAKYLRQKTY 500
IQDIYMKEVD GKLVEGSPDD YTDIKFSVKC AGMTDKIKKE VTFENFKVGF 550
SRKMKPKPVQ VPGGVVLVDD TETIK
SEQ ID NO: 11
MKHMPRKMYS CDFETTTKVE DCRVWAYGYM NIEDHSEYKI GNSLDEFMAW 50
VLKVQADLYF HNLKFDGAFI INWLERNGFK WSADGLPNTY NTIISRMGQW 100
YMIDICIGYK GKRKIHTVIY DSLKKLPFPV KKIAKDFKLT VLKGVIDYHK 150
ERPVGYKITP EEYAYIKNDI QIIAEALKLM FDQGLTRMTA GSDSLKGFKD 200
IITTKKEKKV FPTLSLGLDK EVRKAYRGGF TWLNDKFKEK EIGEGMVEDV 250
NSLYPAQMYS RLLPYGEPIV FEGKYVWDED YPLHIQHIRC EFELKEGYIP 300
TIQIKRSRFY KGNEYLKSSG GEIADLWLSN VDLELLKEHY KLYNVEYVRG 350
LKFKATTGLE KDFIDKWYYI KTHSEGAKKQ LAKLMLNSLY GKFGSNPDVT 400
GKVPYLKENG ALGERLGEEE TKDPVYTPMG VFITAWARYT TITAAQACYD 450
RIIYCDTDSI HLTGTEIPDV IKDIVDPKKL GYWAHESTEK RAKYLRQKTY 500
IQDIYMKEVD GKLVEGSPDD YTDIKFSVKC AGMTDKIKKE VTFENFKVGF 550
SRKMKPKPVQ VPGGVVLVDD TETIK
SEQ ID NO: 12
MKHMPRKMYS CDFETTTKVE DCRVWAYGYM NIEDHSEYKI GNSLDEFMAW 50
VLKVQADLYF HNLKEDGAFI INWLERNGFK WSADGLPNTY NTIISRMGQW 100
YMIDICIGYK GKHKIHTVIY DSLKKLPFPV KKIAKDEKLT VLKGDIDYLK 150
ERPVGYKITP EEYAYIKNDI LIVAEALKLM FDQGLDRMTA GSDSLKGFKD 200
IITTKKFKKV FPTLSLGLDK EVRKAYRGGF TWLNDKFKEK EIGEGMVEDV 250
NSLYPAQMYS RLLPYGEPIV FEGKYVWDED YPLHIQHIRC EFELKEGYIP 300
TIQIKRSREY KGNEYLKSSG GEIADLWLSN VDLELLKEHY KLYNVEYVRG 350
LKFKATTGLE KDFIDKWYYI KTHSEGAKKQ LAKLMLNSLY GKFGSNPDVT 400
GKVPYLKENG ALGFRLGEEE TKDPVYTPMG VFITAWARYT TITAAQACYD 450
RIIYCDTDSI HITGTEIPDV IKDIVDPKKL GYWAHESTEK RAKYIRQKTY 500
IQDIYMKEVD GKIVEGSPDD YTDIKFSVKC AGMTDKIKKE VTFENFKVGF 550
SRKMKPKPVQ VPGGVVLVDD TFTIK
SEQ ID NO: 13
MKHMPRKMYS CDFETTIKVE DCRVWAYGYM NIEDHSEYKI GNSLDEFMAW 50
VLKVQADLYF HNLKFDGAFI INWLERNGFK WSADGLPNTY NTIISRMGQW 100
YMIDICIGYK GKRKIHTSIY DSLKKLPFPV KKIAKDFKLT VLKGNIDYHK 150
ERPVGYKITP EEYAYIKNDI QIIAEALKIM LDQGLDRMTA GSDSLKGFKD 200
IITTKKEKKV FPTISLGLDK EVRKAYRGGF TWINDKFKEK EIGEGMVEDV 250
NSLYPAQMYS RLLPYGEPIV FEGKYVWDED YPLHIQHIRC EFELKEGYIP 300
TIQIKRSRFY KGNEYLKSSG GEIADLWLSN VDLELLKEHY KLYNVEYVRG 350
LKFKATTGLE KDFIDKWYYI KTHSEGAKKQ LAKLMLNSLY GKFGSNPDVT 400
GKVPYLKENG ALGFRLGEEE TKDPVYTPMG VFITAWARYT TITAAQACYD 450
RIIYCDTDSI HLTGTEIPDV IKDIVDPKKL GYWAHESTEK RAKYLRQKTY 500
IQDIYMKEVD GKLVEGSPDD YTDIKFSVKC AGMTDKIKKE VTFENFKVGF 550
SRKMKPKPVQ VPGGVVLVDD TFTIK
SEQ ID NO: 14
MKHMPRKMYS CDFETTTKVE DCRVWAYGYM NIEDHSEYKI GNSLDEFMAW 50
VLKVQADLYF HNLKFDGAFI INWLERNGFK WSADGLPNTY NTIISRMGQW 100
YMIDICIGYK GKRKIHTSIY DSLKKLPFPV KKIAKDFKLT VLKGDIDYHK 150
ERPVGYKITP EEYAYIKNDI QIIAEALKLM LDQGLDRMTA GSDSLKGFKD 200
IITTKKFKKV FPTLSLGLDK EVRKAYRGGF TWLNDKFKEK EIGEGMVFDV 250
NSLYPAQMYS RLLPYGEPIV FEGKYVWDED YPLHIQHIRC EFELKEGYIP 300
TIQIKRSREY KGNEYLKSSG GEIADLWLSN VDLELLKEHY KLYNVEYVRG 350
LKFKATTGLF KDFIDKWYYI KTHSEGAKKQ LAKLMLNSLY GKFGSNPDVT 400
GKVPYLKENG ALGERLGEEE TKDPVYTPMG VFITAWARYT TITAAQACYD 450
RIIYCDTDSI HLTGTEIPDV IKDIVDPKKL GYWAHESTEK RAKYLRQKTY 500
IQDIYMKEVD GKIVEGSPDD YTDIKFSVKC AGMTDKIKKE VTFENFKVGF 550
SRKMKPKPVQ VPGGVVLVDD TFTIK

1. A DNA polymerase, wherein the DNA polymerase has an amino acid sequence comprising at least 80% sequence identity with SEQ ID NO: 1, comprising one or more amino acid substitution wherein the one or more amino acid substitution is selected from the group consisting of A83S, N91I, R96H, R96C, M97V, G108D, G111V, R113H, V118S, S122N, L126I, P127L, A134T, D145V, D145N, D147V, H149R, H149L, Q171L, Q171S, 1173V, L178K, 1179L, Q180M, F181L, K182D, D186T, G197S, D200K, S215P, K220N, V222L, W232Y, M336L, D341K, D341I, D341L, S349R, S349G, T368Y, D398I, V399R, Q560H, and any combination of two or more of the foregoing, wherein the DNA polymerase exhibits polymerase activity.

2. The DNA polymerase of claim 1, wherein the one or more amino acid substitution is selected from the group consisting of A83S, N91L, R96H, R96C, M97V, G108D, G111V, R113H, S122N, P127L, A134T, D145N, D145V, D147V, H149R, H149L, Q180M, D186T, G197S, D341L, T368Y, and any combination of two or more of the foregoing.

3. The DNA polymerase of claim 1, further comprising one or more additional amino acid substitution, wherein the one or more additional amino acid substitution is selected from the group consisting of A83V, N91S, M97K, L107I, K110E, I115L, L123M, L123H, K131E, K138C, K138Q, I172V, G191A, G197D, G197E, Y224K, F230Y, T231V, R236K, F237Y, E239G, 1348V, T368M, T368F, Y369R, T372E, T373H, I378K, K379R, A394G, and any combination of two or more of the foregoing.

4. The DNA polymerase of claim 1, further comprising one or more additional amino acid substitution, wherein the one or more additional amino acid substitution is selected from the group consisting of A83V, N91S, M97K, L107I, K110E, I115L, L123M, K131E, K138Q, G197D, G197E, Y224K, F237Y, T368F, K379R, A394G, and any combination of two or more of the foregoing.

5. The DNA polymerase of claim 1, wherein the amino acid sequence comprises at least 85% sequence identity with SEQ ID NO: 1, at least 90% sequence identity with SEQ ID NO: 1, at least 95% sequence identity with SEQ ID NO: 1, at least 96% sequence identity with SEQ ID NO: 1, at least 97% sequence identity with SEQ ID NO: 1, at least 98% sequence identity with SEQ ID NO: 1, or at least 99% sequence identity with SEQ ID NO: 1

6. The DNA polymerase of claim 1, wherein the amino acid sequence of the DNA polymerase is selected from SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 14.

7. The DNA polymerase of claim 1 wherein the DNA polymerase exhibits one or more of higher yield, higher thermostability, higher solubility, and any combination of two or more of the foregoing relative to a DNA polymerase having the amino acid sequence of SEQ ID NO: 1.

8. A polynucleotide encoding the DNA polymerase of claim 1.

9. A vector comprising the polynucleotide of claim 8.

10. The vector of claim 9, comprising a plasmid, cosmid, a bacterial artificial chromosome, or a phage vector.

11. A kit, comprising the DNA polymerase of claim 1 and a reagent selected from the group consisting of a buffer, deoxyribonucleotides, and any two or more of the foregoing.

12. A method of DNA amplification, comprising synthesizing polynucleotides complementary to a template strand by contacting the template strand with the DNA polymerase of claim 1.

13. The method of claim 12, wherein the amplification comprises multiple displacement amplification, whole genome amplification, plasmid amplification, viral amplification, rolling circle amplification, or preparation of a polynucleotide library for sequencing.

14. A method comprising sequencing one or more of the polynucleotides of claim 12.

15. A kit, comprising the polynucleotide of claim 8 and a reagent selected from the group consisting of a buffer, deoxyribonucleotides, and any two or more of the foregoing.

16. A kit, comprising the vector of claim 9 and a reagent selected from the group consisting of a buffer, deoxyribonucleotides, and any two or more of the foregoing.
